(19) United States
(12) Patent Application Publication
(10) Pub. No.: US 2013/0252839 A1
(43) Pub. Date: Sep. 26, 2013

(54) MARKERS OF PRIMARY GRAFT DYSFUNCTION

(71) Applicants: Sahar Elhanan, Tel Aviv (IL); Eytan Domany, Rehovot (IL); Irun R. Cohen, Rehovot (IL); Peter Hagedorn, Horsholm (DK); Christopher Malcolm Burton, Kgs Lyngby (DK); Henrik Flyvbjerg, Charlottenlund (DK); Martin P. Iversen, Copenhagen O (DK)

(72) Inventors: Sahar Elhanan, Tel Aviv (IL); Eytan Domany, Rehovot (IL); Irun R. Cohen, Rehovot (IL); Peter Hagedorn, Horsholm (DK); Christopher Malcolm Burton, Kgs Lyngby (DK); Henrik Flyvbjerg, Charlottenlund (DK); Martin P. Iversen, Copenhagen O (DK)

(21) Appl. No.: 13/879,877

(22) PCT Filed: Oct. 11, 2011
(86) PCT No.: PCT/IL11/00815

§ 371 Date: Jun. 5, 2013

Related U.S. Application Data
(60) Provisional application No. 61/393,904, filed on Oct. 17, 2010.

(51) Int. Cl. G01N 33/68

(52) U.S. Cl. CPC G01N 33/6884
USPC 506/9; 506/18

(57) ABSTRACT

The present invention relates to methods for diagnosing transplant rejection, or a condition associated with transplant rejection, such as, primary graft dysfunction in a subject, to antigen probe arrays for performing such a diagnosis, and to antigen probe sets for generating such arrays.

### FIELD OF THE INVENTION

The present invention relates to methods for diagnosing or prognosing organ transplant rejection, particularly, primary graft dysfunction in a subject, to antigen probe arrays for performing such a diagnosis, and to antigen probe sets for generating such arrays.

### BACKGROUND OF THE INVENTION

Over the past five decades transplantation has become the primary method of care for patients with end-stage organ failure. While the number of individuals on the waiting list to receive an organ donation has increased exponentially the demand has not been met due to difficulties with organ procurement and the immunological rejection response. Those individuals who are lucky enough to undergo organ transplantation are still faced with many challenges, such as graft rejection.

Primary graft dysfunction (PGD) is a form of ischemia-reperfusion injury occurring in the early period following transplantation, and is most often seen in the transplanted lung, liver, or kidney and can lead to graft rejection. Patients with PGD have markedly worse 90-day post-operative mortality and 3-year survival (Arcasoy et al., 2005).

The incidence of PGD after lung, kidney and heart transplant is estimated as 20%, 24% and 20%, respectively, and it is considered a significant cause of morbidity and mortality after solid-organ transplantation. A recent report indicated an association for the development of PGD across different organs retrieved and transplanted from the same donor (Oto et al., 2008).

In lung transplanted patients, PGD is observed by development of pulmonary infiltrates and impaired oxygenation within the first 3 days after lung transplantation, (Christie et al., 2005). The specific aetiology and pathogenesis of PGD is not well understood but is thought to be the result of complex interactions between donor lung and recipient immune system (Lee and Christie, 2009). Injuries to pulmonary epithelium and endothelium by reactive oxygen species, initiation of aggressive inflammatory cascades, and increases in pro-coagulant and vasoconstriction factors have all been implicated (Pelaez et al., 2010; Salama et al., 2010).

Autoimmunity, specifically T-cell autoreactivity towards type V collagen (COLS), has been associated with the development of PGD (Bobadilla et al., 2008). It is well established that reactivity towards this protein is also associated with the development of obliterative bronchiolitis (Sumpter and Wilkes, 2004). Recently, the autoantibody repertoires in the blood of recipients at various stages of chronic lung rejection in the form of obliterative bronchiolitis were studied using an antigen microarray containing hundreds of self-molecules (Hagedorn et al., 2010). It was found that a profile of autoantibodies binding to 28 proteins or their peptides could differentiate between mild and severe chronic rejection. Comparing donor lungs developing PGD with those that did not has identified significantly different expression for hundreds of genes involved in both signaling and stress-activated pathways (Ray et al., 2007; Anraku et al., 2008).

Antigen microarrays are newly developed tools for the high-throughput characterization of the immune response, and have been used to analyze immune responses in vaccination and in autoimmune disorders. Autoimmune repertoires analyses of human health and disease conditions showed different patterns of multiple reactivities, indicating that multiple reactivities are more revealing than single antigen-antibody relationships (Quintana et al., 2006; Merbl et al., 2007). Thus, autoantibody repertoires have the potential to provide both new insights into the pathogenesis of the disease and to serve as immune biomarkers (Cohen, 2007) of the disease process.

Antigen microarrays have been used to characterize serum autoantibodies in systemic lupus erythematosus (Li et al., 2005), rheumatoid arthritis (Hueber et al., 2005) and neuromyelitis optica (Lalive et al. 2006).

PCT Pub. No. WO 02/08755 to some of the inventors of the present invention is directed to a method, system and an article of manufacture for clustering and thereby identifying predefined antigens reactive with undetermined immunoglobulins of sera derived from patient subjects in need of diagnosis of disease or monitoring of treatment. The '755 publication discloses the use of antigen arrays for identifying antigens reactive with immunoglobulins of sera derived from subjects afflicted with various diseases.

U.S. Pat. App. Pub. No. 2005/0260770 to some of the inventors of the present invention discloses a method of diagnosing an immune disease or a predisposition thereto in a subject, comprising determining a capacity of immunoglobulins of the subject to specifically bind each antigen probe of an antigen probe set. The antigen probe set comprises a plurality of antigen probes selected from the group consisting of at least a portion of a cell/tissue structure molecule, at least a portion of a heat shock protein, at least a portion of an immune system molecule, at least a portion of a homopolymeric polypeptide, at least a portion of a hormone, at least a portion of a metabolic enzyme, at least a portion of a microbial antigen, at least a portion of a molluscan antigen, at least a portion of a nucleic acid, at least a portion of a plant antigen, at least a portion of a plasma molecule, and at least a portion of a tissue antigen, wherein the binding capacity of the immunoglobulin of the subject is indicative of the immune disease or the predisposition thereto.

U.S. Pat. App. Pub. No 2007/0218482 relates to a method of screening for, diagnosing or detecting risk of primary graft failure, comprising the steps: (a) determining the level of RNA product of one or more biomarkers selected from a biomarkers set in a sample from a donor lung; and (b) comparing the level of RNA products in the sample with a control, wherein detecting differential expression of the RNA products between the donor lung and the control is indicative of risk for primary graft failure.

U.S. Pat. App. Pub. No 2006/0105345 provides a method for diagnosing lung transplantation rejection comprising determining the amount of hepatocyte growth factor (HGF) in a body fluid or tissue sample of a patient who has undergone lung transplantation.

U.S. Pat. App. Pub. No 2007/0134728 relates to methods of diagnosing, predicting and monitoring conditions and disorders associated organ transplantation and organ health. In particular, the '728 publication relates to the diagnosis, prediction and monitoring of disorders, conditions, and organ status by detection of cytokines, cytokine-related compounds, and chemokines, particularly in urine. The '728 publication further relates to methods and compositions for assessing the efficacy of agents and interventions used to treat organ associated disorders and conditions and for maintaining organ health.

However, none of the prior art discloses an antigen array that can provide a specific, reliable, accurate and discriminatory assay for diagnosing conditions or disorders associated with organ transplant rejection, particularly primary graft dysfunction, including but not limited to, in lung, heart, kidney and liver recipients.

Thus, there remains a need for improved diagnostic methods and kits useful in diagnosing primary graft dysfunction in a subject.

### SUMMARY OF THE INVENTION

The present invention provides methods and kits for diagnosing organ transplant rejection in a subject. In particular, the invention provides methods and kits for diagnosing primary graft dysfunction (PGD) in a subject, antigen probe arrays for practicing such a diagnosis, and antigen probe sets for generating such arrays. The present invention provides unique antigen-autoantibody reactivity patterns relevant to organ transplantation rejection or a condition or disorder associated with organ transplantation, particularly primary graft dysfunction.

Table 1 Lists the Antigens Having Increased Reactivity in Subjects with PGD

| | | | SEQ |
|---|---|---|---|
| Antigen | Gene Name | EntrezID | ID NO: |
| TEP1 | telomerase-associated protein 1 | 7011 | 1 |
| EGFR | epidermal growth factor receptor | 1956 | 2 |
| MBP | myelin basic protein | 4155 | 3 |
| MLANA | melan-A | 2315 | 4 |
| MUC1 | mucin 1, cell surface associated | 4582 | 5 |
| MYCL1 | v-myc myelocytomatosis viral | 4610 | 6 |
| | oncogene 1 | | |
| PLCG1 | phospholipase C, gamma 1 | 5335 | 7 |
| RB1 | retinoblastoma 1 (including | 5925 | 8 |
| | osteosarcoma) | | |
| CERK | ceramide kinase | 64781 | 9 |
| CYP3A4 | cytochrome P450, 3A4 | 1576 | 10 |
| SOCS3 | suppressor of cytokine signaling 3 | 9021 | 11 |
| PRKCA | protein kinase C, alpha | 5578 | 12 |
| HSP90AA1 | heat shock protein 90 kDa alpha, A1 | 3320 | 13 |
| IGF1R | insulin-like growth factor 1 | 3480 | 14 |
| | receptor | | |
| HSPD1 | heat shock 60 kDa protein 1 | 3329 | 15 |
| | (chaperonin) | | |
| TARP | TCR gamma alt. reading frame | 445347 | 16 |
| | protein | | |
| TP53 | tumor protein p53 | 7157 | 17 |

It is now disclosed for the first time that exemplary lung transplant recipients manifest IgG and IgM autoantibody reactivity, and that specific patterns of reactivity to self-antigens discriminate between patients with and without PGD. The unique PGD signature pattern predicted the PGD grade of an independent patient cohort with remarkably high sensitivity and specificity.

According to a first aspect, the present invention provides a method of diagnosing primary graft dysfunction in a subject in need thereof, the method comprising determining the reactivity of antibodies in a sample obtained from the subject to a plurality of antigens selected from the group consisting of: TEP1, EGFR, MBP, MLANA, MUC1, MYCL1, PLCG1, RB1, CERK, CYP3A4, SOC3, PRKCA, HSP90AA1, IGF1R, HSPD1, TARP and TP53, thereby determining the reactivity pattern of the sample to the plurality of antigens, and comparing said reactivity pattern of the sample to a control reactivity pattern, wherein a significant difference between said reactivity pattern of the sample compared to the control reactivity pattern is an indication that the subject is afflicted with primary graft dysfunction.

According to another embodiment, the plurality of antigens comprises at least three different antigens. According to another embodiment, the plurality of antigens comprises at least four different antigens. According to another embodiment, the plurality of antigens comprises at least five different antigens. According to another embodiment, the plurality of antigens comprises at least ten different antigens. According to another embodiment, the plurality of antigens comprises at least fifteen different antigens. According to another embodiment, the plurality of antigens comprises at least sixteen different antigens.

According to another embodiment, the plurality of antigens comprises TEP1, EGFR, MBP, MLANA, MUC1, MYCL1, PLCG1, RB1, CERK, CYP3A4, SOC3, PRKCA, HSP90AA1, IGF1R, HSPD1, TARP and TP53. According to another embodiment, the plurality of antigens consists of TEP1, EGFR, MBP, MLANA, MUC1, MYCL1, PLCG1, RB1, CERK, CYP3A4, SOC3, PRKCA, HSP90AA1, IGF1R, HSPD1, TARP and TP53.

According to another embodiment, the plurality of antigens comprises no more than 17 antigens. According to another embodiment, the plurality of antigens comprises no more than 20 antigens. According to another embodiment, the plurality of antigens comprises no more than 25 antigens. According to another embodiment, the plurality of antigens comprises no more than 30 antigens. According to another embodiment, the plurality of antigens comprises no more than 40 antigens. According to another embodiment, the plurality of antigens comprises no more than 50 antigens. Each possibility represents a separate embodiment of the invention.

As used herein, the “reactivity of antibodies in a sample” to “a plurality of antigens” refers to the immune reactivity of each antibody in the sample to a specific antigen selected from the plurality of antigens. The immune reactivity of the antibody to the antigen, i.e. its ability to specifically bind the antigen, may be used to determine the amount of the antibody in the sample. The reactivity pattern of the sample thus reflects the levels of each one of the tested antibodies in the sample.

Typically, determining the reactivity of antibodies in the sample to the plurality of antigens is performed using an immunoassay. Advantageously, the plurality of antigens may be used in the form of an antigen array. According to some embodiments the antigen array is arranged in the form of an antigen chip.

A “significant difference” between reactivity patterns refers, in different embodiments, to a statistically significant difference, or in other embodiments to a significant difference as recognized by a skilled artisan. Advantageously, the methods of the invention may employ the use of learning and pattern recognition analyzers, clustering algorithms and the like, in order to discriminate between reactivity patterns of samples obtained from subjects having a condition associated with organ transplant rejection (e.g., PGD following graft transplantation) to control samples. As such, this term specifically includes a difference measured by, for example, determining the reactivity of antibodies in a test sample to a plurality of antigens, and comparing the resulting reactivity pattern to the reactivity patterns of negative and/or positive control samples (e.g., samples obtained from the patients prior to the transplantation procedure, or samples obtained from control subjects which did not develop PGD following organ transplantation or subjects which developed PGD, respectively) using such algorithms and/or analyzers. The difference may also be measured by comparing the reactivity pattern of the test sample to a predetermined classification rule or threshold obtained in such manner. Thus, in another embodiment, a significant difference between the reactivity pattern of a test sample compared to a reactivity pattern of a control sample, wherein the difference is computed using a learning and pattern recognition algorithm, indicates that the subject is afflicted with a condition associated with organ transplant rejection (e.g., PGD).

As used herein, the term “primary graft dysfunction” relates to a form of ischemia-reperfusion injury occurring in the early period following transplantation. As known to the ordinarily skilled artisan, PGD, also termed severe ischemia-reperfusion injury, early graft dysfunction or the re-implantation response, is most often seen in the transplanted lung, liver, or kidney and can lead to graft rejection. According to some embodiments, the graft is selected from the group consisting of: lung, heart, kidney and liver. According to a particular embodiment, the graft is a lung.

According to another aspect, the present invention provides a method for diagnosing a condition associated with organ transplantation rejection in a subject in need thereof, the method comprising determining the reactivity of antibodies in a sample obtained from the subject to a plurality of antigens selected from the group consisting of: TEP1, EGFR, MBP, MLANA, MUC1, MYCL1, PLCG1, RB1, CERK, CYP3A4 and SOC3, thereby determining the reactivity pattern of the sample to the plurality of antigens, and comparing said reactivity pattern of the sample to a control reactivity pattern, wherein a significant difference between said reactivity pattern of the sample compared to the control reactivity pattern is an indication that the subject is afflicted a condition associated with organ transplantation rejection.

According to some embodiments, said organ is selected from the group consisting of: lung, heart, kidney and liver. According to a particular embodiment, said organ is lung.

According to a specific embodiment, the condition associated with organ transplantation rejection is primary graft dysfunction.

According to another embodiment, the plurality of antigens comprises at least 3 different antigens. According to another embodiment, the plurality of antigens comprises at least 4 different antigens. According to another embodiment, the plurality of antigens comprises at least 5 different antigens. According to another embodiment, the plurality of antigens comprises TEP1, EGFR, MBP, MLANA, MUC1, MYCL1, PLCG1, RB1, CERK, CYP3A4 and SOC3. According to another embodiment, the plurality of antigens further comprises at least one antigen selected from the group consisting of: PRKCA, HSP90AA1, IGF1R, HSPD1, TARP and TP53. According to another embodiment, the plurality of antigens further comprises PRKCA, HSP90AA1, IGF1R, HSPD1, TARP and TP53.

According to some embodiments of the methods of the invention, the antibodies are selected from IgG and IgM antibodies. According to another embodiment, the reactivity pattern comprises at least one IgG reactivity. According to yet another embodiment, the reactivity pattern comprises at least one IgM reactivity. According to yet another embodiment, the reactivity pattern comprises at least one IgG reactivity and at least one IgM reactivity.

According to additional embodiments of the methods of the invention, the control is selected from the group consisting of a sample from at least one individual, a panel of control samples from a set of individuals, and a stored set of data from control individuals.

According to another embodiment of the methods of the invention, the control reactivity pattern is obtained from said subject before undergoing organ transplantation. According to another embodiment, the control reactivity pattern is obtained from healthy subjects. According to another embodiment, the control reactivity pattern is obtained from subjects who did not develop primary graft dysfunction. According to another embodiment, the control reactivity pattern is obtained from subjects who did not develop a condition associated with organ transplantation rejection.

According to another embodiment of the methods of the invention, the sample is a fluid sample. According to another embodiment, the sample is a blood sample. According to another embodiment, the sample is a serum sample.

The plurality of antigens, according to another embodiment of the methods of the invention, is used in the form of an antigen array.

According to another aspect, the present invention provides a kit for the diagnosis primary graft dysfunction comprising a plurality of antigens selected from the group consisting of TEP1, EGFR, MBP, MLANA, MUC1, MYCL1, PLCG1, RB1, CERK, CYP3A4, SOC3, PRKCA, HSP90AA1, IGF1R, HSPD1, TARP and TP53.

According to another aspect, the present invention provides a kit for the diagnosis a condition associated with organ transplantation rejection comprising a plurality of antigens selected from the group consisting of TEP1, EGFR, MBP, MLANA, MUC1, MYCL1, PLCG1, RB1, CERK, CYP3A4 and SOC3.

According to another embodiment, the kit of the invention is in the form of an antigen array. According to another embodiment, the kit further comprises means for determining the reactivity of antibodies in a sample to the plurality of antigens. According to another embodiment, the kit further comprises means for comparing reactivity patterns of antibodies in different samples to the plurality of antigens.

According to another aspect, the present invention provides an antigen probe set comprising a plurality of antigen probes selected from the group consisting of TEP1, EGFR, MBP, MLANA, MUC1, MYCL1, PLCG1, RB1, CERK, CYP3A4, SOC3, PRKCA, HSP90AA1, IGF1R, HSPD1, TARP and TP53.

According to another aspect, the present invention provides an antigen probe set comprising a plurality of antigen probes selected from the group consisting of TEP1, EGFR, MBP, MLANA, MUC1, MYCL1, PLCG1, RB1, CERK, CYP3A4, SOC3, PRKCA, HSP90AA1, IGF1R, HSPD1, TARP and TP53, for use in diagnosing primary graft dysfunction in a subject in need thereof.

According to another aspect, the present invention provides an antigen probe set comprising a plurality of antigen probes selected from the group consisting of TEP1, EGFR, MBP, MLANA, MUC1, MYCL1, PLCG1, RB1, CERK, CYP3A4 and SOC3.

According to another aspect, the present invention provides an antigen probe set comprising a plurality of antigen probes selected from the group consisting of TEP1, EGFR, MBP, MLANA, MUC1, MYCL1, PLCG1, RB1, CERK, CYP3A4 and SOC3, for use in diagnosing a condition associated with organ transplantation rejection in a subject in need thereof.

Other objects, features and advantages of the present invention will become clear from the following description and drawings.

### BRIEF DESCRIPTION OF THE DRAWINGS

FIG. 1. Concordance between IgG and IgM reactivity changes

FIG. 2. Distributions of autoreactivities including both BOS and PGD status. Autoreactivities (log₂ transformed and normalized to the median) from the 39 patients were grouped according to both BOS and PGD status and the distribution within each group presented as a boxplot, for the 6 antigens also identified in Hagedorn et al., 2010.

FIG. 3. PGD network. Network of the 12 differentially reactive proteins that interact directly. Biological themes summarizing overrepresented biological processes in the network are indicated. The 5 differentially reactive proteins not in the network are also shown for completeness.

FIG. 4. Classification and Prediction of PGD status. A) The 17 proteins identified were used for PGD class prediction in the training set using a nearest centroid (NC) classification algorithm. B) The trained NC classifier was then used for PGD class prediction in the validation set. Results are shown in modified 2×2 contingency tables that were used to calculate the percentage of classifications that agreed with clinical diagnosis. P-values were calculated with Fisher's exact test.

FIG. 5 Correlation between reactivity and expression changes. A) Scatterplot between gene expression changes (GSE8021 study) and IgM reactivity changes. B) Scatterplot between gene expression changes measured in both mRNA studies. The Pearson correlation coefficient and its associated P-value are shown for each scatter.

### DETAILED DESCRIPTION OF THE INVENTION

The present invention provides methods of diagnosing a disorder or condition associated with organ transplant rejection (e.g., PGD) in a subject, using antigen probe arrays for practicing such a diagnosis, and identifies specific antigen probe sets for generating such arrays. According to some embodiments, the present invention relates to an autoantibody-based biomarker test for diagnosis of primary graft dysfunction including but not limited to after lung transplantation.

The present invention is based in part on the unexpected results obtained when testing the antibody reactivity of lung transplant recipients using an antigen array. As exemplified herein below, lung transplant recipients manifest IgG and IgM autoantibody reactivity, and specific patterns of reactivity to self-antigens discriminate between patients with and without PGD.

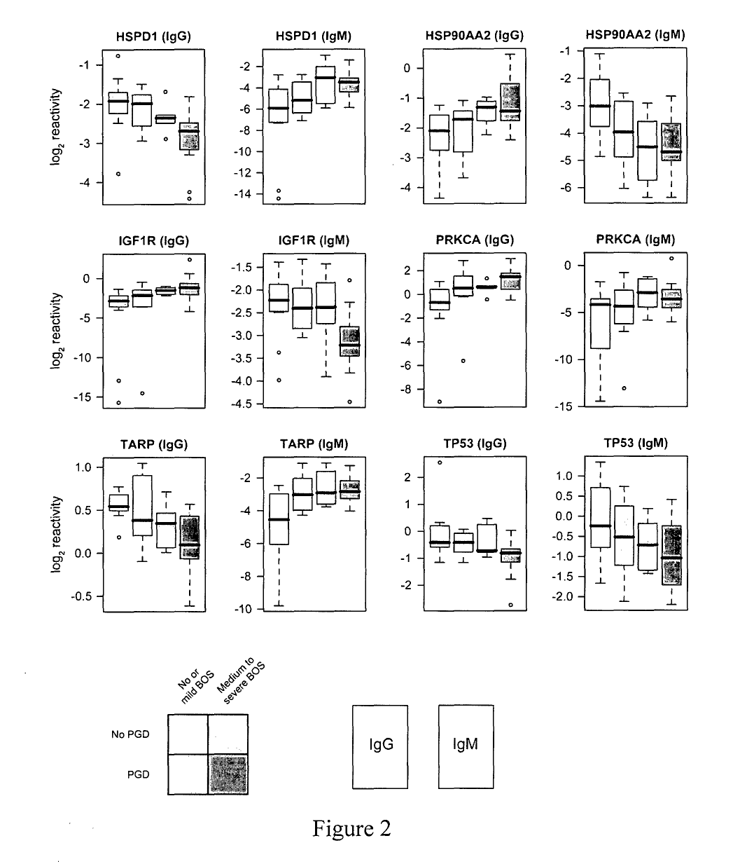

Whether PGD may induce or accelerate chronic rejection in the form Bronchiolitis Obliterans (BO) has been debated and conflicting results have been published (Arcasoy et al., ibid.). As exemplified herein below, no significant correlation between BOS and PGD grades was observed among the 39 patients included in the study (Table 2). However, 6 (35%) out of the 17 informative proteins were also observed to be informative with respect to BOS (Hagedorn et al. ibid.). A two-factor ANOVA including both BOS and PGD as factors in general confirms the significant differential reactivity with respect to both factors (Table 4 and FIG. 2).

According to some embodiments, the present invention provides a method of diagnosing a condition associated with organ transplantation rejection, particularly, primary graft dysfunction, in a subject in need thereof, the method comprising determining the reactivity of antibodies in a sample obtained from the subject to a plurality of antigens selected from the group consisting of: TEP1, EGFR, MBP, MLANA, MUC1, MYCL1, PLCG1, RB1, CERK, CYP3A4, SOC3, PRKCA, HSP90AA1, IGF1R, HSPD1, TARP and TP53, thereby determining the reactivity pattern of the sample to the plurality of antigens, and comparing said reactivity pattern of the sample to a control reactivity pattern, wherein a significant difference between said reactivity pattern of the sample compared to the control reactivity pattern is an indication that the subject is afflicted with a condition associated with organ transplantation rejection.

According to another embodiment, the present invention provides a method for diagnosing a condition associated with organ transplantation rejection in a subject in need thereof, the method comprising determining the reactivity of antibodies in a sample obtained from the subject to at least seven antigens selected from the group consisting of: TEP1, EGFR, MBP, MLANA, MUC1, MYCL1, PLCG1, RB1, CERK, CYP3A4, SOC3, PRKCA, HSP90AA1, IGF1R, HSPD1, TARP and TP53, thereby determining the reactivity pattern of the sample to the plurality of antigens, and comparing said reactivity pattern of the sample to a control reactivity pattern, wherein a significant difference between said reactivity pattern of the sample compared to the control reactivity pattern is an indication that the subject is afflicted a condition associated with organ transplantation rejection.

Antigen Probes and Antigen Probe Sets

According to further embodiments, the invention provides antigen probes and antigen probe sets useful for diagnosing a disorder or condition associated with organ transplant rejection (e.g., PGD), as detailed herein.

According to the principles of the invention, the invention further provides a plurality of antigens also referred to herein as antigen probe sets. These antigen probe sets comprising a plurality of antigens are reactive specifically with the sera of subjects having a disorder or condition associated with organ transplant rejection. According to the principles of the invention, the plurality of antigens may advantageously be used in the form of an antigen array. According to some embodiments the antigen array is conveniently arranged in the form of an antigen chip.

A “probe” as used herein means any compound capable of specific binding to a component. According to one aspect, the present invention provides an antigen probe set comprising a plurality of antigens selected from the group consisting of: TEP1, EGFR, MBP, MLANA, MUC1, MYCL1, PLCG1, RB1, CERK, CYP3A4, SOC3, PRKCA, HSP90AA1, IGF1R, HSPD1, TARP and TP53. According to certain embodiments, the antigen probe set comprises a subset of the antigens of the present invention. In a particular embodiment, the subset of antigen consists of TEP1, EGFR, MBP, MLANA, MUC1, MYCL1, PLCG1, RB1, CERK, CYP3A4 and SOC3. According to another embodiment, the plurality of antigens comprises at least 2, at least 3, at least 4, at least 5, at least 6, at least 7, at least, 8, at least 9, at least 10, at least 11, at least 12, at least 13, at least 14, at least 15 or at least 16 different antigens. Typically, the reactivity of antibodies to the plurality of antigens of the invention are determined according to techniques known in the art.

The antigens used in the present invention are known in the art and are commercially available, e.g., from Sigma Aldrich. Antigen probes to be used in the assays of the invention may be purified or synthesized using methods well known in the art. For example, an antigenic protein or peptide may be produced using known recombinant or synthetic methods, including, but not limited to, solid phase (e.g. Boc or f-Moc chemistry) and solution phase synthesis methods (Stewart and Young, 1963; Meienhofer, 1973; Schroder and Lupke, 1965; Sambrook et ah, 2001). One of skill in the art will possess the required expertise to obtain or synthesize the antigen probes of the invention.

It should be noted, that the invention utilizes antigen probes as well as homologs, fragments and derivatives thereof, as long as these homologs, fragments and derivatives are immunologically cross-reactive with these antigen probes. The term “immunologically cross-reactive” as used herein refers to two or more antigens that are specifically bound by the same antibody.

The antigenic proteins polypeptides of the invention are listed in Table 1 above, including their Gene ID No. as well as an exemplary amino acid sequence. As known to one skilled in the art a single gene may have several variants encoding distinct isoforms. It should be appreciated that the present invention encompasses transcript variants in addition to those mentioned in Table 1 (SEQ ID NO:1-17). Thus, in some embodiments, the TP53 antigen has an amino acid selected from NP_{—}000537.3 (SEQ ID NO: 17), NP_{—}001119584.1, NP_{—}001119585.1, NP_{—}001119586.1, NP_{—}001119587.1, NP_{—}001119588.1 and NP_{—}001119589.1. In another embodiment the TARP antigen has an amino acid selected from NP_{—}001003799.1 (SEQ ID NO: 16) and NP_{—}001003806.1. In another embodiment the HSPD1 antigen has an amino acid selected from NP_{—}002147.2 (SEQ ID NO: 15) and NP_{—}955472.1. In another embodiment the HSP90AA1 antigen has an amino acid selected from NP_{—}001017963.2 (SEQ ID NO: 13) and NP_{—}005339.3. In another embodiment the CYP3A4 antigen has an amino acid selected from NP_{—}059488.2 (SEQ ID NO: 10) and NP_{—}001189784.1. In another embodiment the PLCG1 antigen has an amino acid selected from NP_{—}002651.2 (SEQ ID NO: 7) and NP_{—}877963.1. In another embodiment the MYCL1 antigen has an amino acid selected from NP_{—}001028253.1 (SEQ ID NO: 6), NP_{—}001028254.2 and NP_{—}005367.2. In another embodiment the MUC1 antigen has an amino acid selected from NP_{—}002447.4 (SEQ ID NO: 5), NP_{—}001018016.1, NP_{—}001018017.1, NP_{—}001037855.1, NP_{—}001037856.1, NP_{—}001037857.1, NP_{—}001037858.1, NP_{—}001191214.1, NP_{—}001191215.1, NP_{—}001191216.1, NP_{—}001191217.1, NP_{—}001191218.1, NP_{—}001191219.1, NP_{—}001191220.1, NP_{—}001191221.1, NP_{—}001191222.1, NP_{—}001191223.1, NP_{—}001191224.1, NP_{—}001191225.1 and NP_{—}001191226.1. In another embodiment the MBP antigen has an amino acid selected from NP_{—}001020252.1 (SEQ ID NO: 3), NP_{—}001020261.1, NP_{—}001020263.1, NP_{—}001020271.1, NP_{—}001020272.1 and NP_{—}002376.1. In another embodiment the EGFR antigen has an amino acid selected from NP_{—}005219.2 (SEQ ID NO: 2), NP_{—}958439.1, NP_{—}958440.1 and NP_{—}958441.1. Each possibility represents a separate embodiment of the present invention.

The term “homolog” as used herein refers to a peptide which having at least 70%, at least 75%, at least 80%, at least 85% or at least 90% identity to the antigen's amino acid sequence. Cross-reactivity can be determined by any of a number of immunoassay techniques, such as a competition assay (measuring the ability of a test antigen to competitively inhibit the binding of an antibody to its known antigen).

The term “fragment” as used herein refers to a portion of a polypeptide, or polypeptide analog which remains immunologically cross-reactive with the antigen probes, e.g., to immunospecifically recognize the target antigen. The fragment may have the length of about 5%, about 10%, about 20%, about 40%, about 50%, about 60%, about 70%, about 80%, about 85%, about 90% or about 95% of the respective antigen.

The term peptide typically refers to a polypeptide of up to about 50 amino acid residues in length. According to particular embodiments, the antigenic peptides of the invention may be 10-50 amino acids in length and are typically about 10-30 or about 15-25 amino acids in length. According to yet another particular embodiment, the reactivity of a single antibody of the invention may be assayed using more than one antigen.

The term peptides encompasses native peptides (either degradation products, synthetically synthesized peptides, or recombinant peptides), peptidomimetics (typically, synthetically synthesized peptides), and the peptide analogues peptoids and semipeptoids, and may have, for example, modifications rendering the peptides more stable while in a body or more capable of penetrating into cells. Such modifications include, but are not limited to N-terminus modifications; C-terminus modifications; peptide bond modifications, including but not limited to CH₂—NH, CH₂—S, CH₂—S=0, 0=C—NH, CH₂—O, CH₂—CH₂, S═C—NH, CH—CH, and CF═CH; backbone modifications; and residue modifications.

The antigens of the invention may be used having a terminal carboxy acid, as a carboxy amide, as a reduced terminal alcohol or as any pharmaceutically acceptable salt, e.g., as metal salt, including sodium, potassium, lithium or calcium salt, or as a salt with an organic base, or as a salt with a mineral acid, including sulfuric acid, hydrochloric acid or phosphoric acid, or with an organic acid e.g., acetic acid or maleic acid.

Functional derivatives consist of chemical modifications to amino acid side chains and/or the carboxyl and/or amino moieties of said peptides. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those polypeptides, which contain one or more naturally occurring or modified amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and ornithine may be substituted for lysine.

The amino acid residues described herein are in the “L” isomeric form, unless otherwise indicated. However, residues in the “D” isomeric form can be substituted for any L-amino acid residue, as long as the peptide substantially retains the desired antibody specificity.

Suitable analogs may be readily synthesized by now-standard peptide synthesis methods and apparatus or recombinant methods. All such analogs will essentially be based on the antigens of the invention as regards their amino acid sequence but will have one or more amino acid residues deleted, substituted or added. When amino acid residues are substituted, such conservative replacements which are envisaged are those which do not significantly alter the structure or antigenicity of the polypeptide. For example basic amino acids will be replaced with other basic amino acids, acidic ones with acidic ones and neutral ones with neutral ones. In addition to analogs comprising conservative substitutions as detailed above, analogs comprising non-conservative amino acid substitutions are further contemplated, as long as these analogs are immunologically cross reactive with a peptide of the invention.

In other aspects, there are provided nucleic acids encoding these peptides, vectors comprising these nucleic acids and host cells containing them. These nucleic acids, vectors and host cells are readily produced by recombinant methods known in the art (see, e.g., Sambrook et al., 2001). For example, an isolated nucleic acid sequence encoding an antigen of the invention can be obtained from its natural source, either as an entire (i.e., complete) gene or a portion thereof. A nucleic acid molecule can also be produced using recombinant DNA technology (e.g., polymerase chain reaction (PCR) amplification, cloning) or chemical synthesis. Nucleic acid sequences include natural nucleic acid sequences and homologs thereof, including, but not limited to, natural allelic variants and modified nucleic acid sequences in which nucleotides have been inserted, deleted, substituted, and/or inverted in such a manner that such modifications do not substantially interfere with the nucleic acid molecule's ability to encode a functional peptide of the present invention.

Diagnostic Methods

According to some embodiments, the invention provides diagnostic methods useful for the detection of a disorder or condition associated with organ transplant rejection, particularly PGD. In one embodiment the subject is a mammal, preferably a human.

As used herein the term “diagnosing” or “diagnosis” refers to the process of identifying a medical condition or disorder (e.g., PGD) by its signs, symptoms, and in particular from the results of various diagnostic procedures, including e.g. detecting the reactivity of antibodies in a biological sample (e.g. serum) obtained from an individual, to a plurality of antigens. Furthermore, as used herein the term “diagnosing” or “diagnosis” encompasses screening for a disorder, detecting a presence or a severity of a disorder, distinguishing a disorder from other disorders including those that may feature one or more similar or identical symptoms, providing prognosis of a disease, monitoring disease progression or relapse, as well as assessment of treatment efficacy and/or relapse of a disorder or condition, as well as selecting a therapy and/or a treatment for a disorder, optimization of a given therapy for a disorder, monitoring the treatment of a disorder, and/or predicting the suitability of a therapy for specific patients or subpopulations or determining the appropriate dosing of a therapeutic product in patients or subpopulations. In one embodiment, the subject being diagnosed according to the methods of the invention is symptomatic. In other embodiments, the subject is asymptomatic.

According to some embodiments, the methods of the invention are effected by determining the reactivity of antibodies in a sample obtained from a test subject to a plurality of antigens selected from the group consisting of: TEP1, EGFR, MBP, MLANA, MUC1, MYCL1, PLCG1, RB1, CERK, CYP3A4, SOC3, PRKCA, HSP90AA1, IGF1R, HSPD1, TARP and TP53, thereby determining the reactivity pattern of the sample to the plurality of antigens, and comparing the reactivity pattern of said sample to a control reactivity pattern. In one embodiment, a significant difference between the reactivity pattern of said sample compared to a reactivity pattern of a control sample indicates that the subject is afflicted with a disorder or condition associated with organ transplant rejection, particularly PGD.

As used herein, the “reactivity of antibodies in a sample” to “a plurality of antigens” refers to the immune reactivity of each antibody in the sample to a specific antigen selected from the plurality of antigens. The immune reactivity of the antibody to the antigen, i.e. its ability to specifically bind the antigen, may be used to determine the amount of the antibody in the sample, thereby providing a quantitative assay. In a particular embodiment, the reactivity is quantitatively determined. Thus, for instance, the reactivity of an antibody to an antigen may be increased or decreased. The calculated levels of each one of the tested antibodies in the sample are selectively referred to as the reactivity pattern of the sample to these antigens. For instance, in the Examples below, the reactivity of each antigen was calculated and presented as the scaled mean log intensity of each spot (antigen).

An antibody “directed to” an antigen, as used herein is an antibody which is capable of specifically binding the antigen. Determining the levels of antibodies directed to a plurality of antigens includes measuring the level of each antibody in the sample, wherein each antibody is directed to a specific antigen of the invention. This step is typically performed using an immunoassay, as detailed herein.

In other embodiments, determining the reactivity of antibodies in said sample to said plurality of antigens, (and the levels of each one of the tested antibodies in the sample) is performed by a process comprising:

(i) contacting the sample, under conditions such that a specific antigen-antibody complex may be formed, with an antigen probe set comprising said plurality of antigens, and

(ii) quantifying the amount of antigen-antibody complex formed for each antigen probe.

The amount of antigen-antibody complex is indicative of the level of the tested antibody in the sample (or the reactivity of the sample with the antigen).

In certain embodiments, the test sample and control samples comprise IgG and/or IgM antibodies. Particularly, the test sample and control samples may comprise IgG and IgM antibodies. In yet another preferred embodiment, the test and control samples comprise a plurality of IgG antibodies and a plurality of IgM antibodies. In certain embodiments, the methods of the invention are effected by determining the reactivity of IgG and/or IgM antibodies in a test and control sample to a plurality of antigens. In certain embodiments, the methods of the invention are effected by determining the reactivity of at least one IgG and at least one IgM antibodies in a test and control sample to a plurality of antigens. In another embodiment, the reactivity of at least one antibody to a specific antigen from the plurality of antigens of the invention is up-regulated. In another embodiment, the reactivity of at least one antibody to a specific antigen is down-regulated.

In some embodiments, the methods of the present invention employ an antigen microarray system for informatically characterizing informative patterns of antibodies as specific biomarkers for grading PGD, as detailed herein.

Diagnostic methods differ in their sensitivity and specificity. The “sensitivity” of a diagnostic assay is the percentage of diseased individuals (e.g., those who develop PGD) who test positive (percent of “true positives”). Diseased individuals not detected by the assay are “false negatives”. Subjects who are not diseased and who test negative in the assay are termed “true negatives”. The “specificity” of a diagnostic assay is 1 minus the false positive rate, where the “false positive” rate is defined as the proportion of those without the disease who test positive.

In some embodiments, the plurality of antigens is selected to exhibit at least 70%, at least 80%, at least 85%, at least 90%, at least 95%, or at least 99% sensitivity, combined with at least 70%, at least 80%, at least 85%, at least 90%, or at least 95% specificity. In some embodiments, both the sensitivity and specificity are at least 75%, at least 80%, at least 85%, at least 90%, or at least 95%. In an exemplary embodiment, the plurality of antigens is selected to exhibit at least 80% sensitivity, combined with at least 95% specificity.

Antibodies, Samples and Immunoassays

Antibodies, or immunoglobulins (Ig), comprise two heavy chains linked together by disulfide bonds and two light chains, each light chain being linked to a respective heavy chain by disulfide bonds in a “Y” shaped configuration. Each heavy chain has at one end a variable domain (VH) followed by a number of constant domains (CH). Each light chain has a variable domain (VL) at one end and a constant domain (CL) at its other end, the light chain variable domain being aligned with the variable domain of the heavy chain and the light chain constant domain being aligned with the first constant domain of the heavy chain (CHI). The variable domains of each pair of light and heavy chains form the antigen binding site.

The isotype of the heavy chain (gamma, alpha, delta, epsilon or mu) determines immunoglobulin class (IgG, IgA, IgD, IgE or IgM, respectively). The light chain is either of two isotypes (kappa, ic or lambda, 2) found in all antibody classes.

It should be understood that when the terms “antibody” or “antibodies” are used, this is intended to include intact antibodies, such as polyclonal antibodies or monoclonal antibodies (mAbs), as well as proteolytic fragments thereof such as the Fab or F(ab′)2 fragments. Further included within the scope of the invention (for example as immunoassay reagents, as detailed herein) are chimeric antibodies; recombinant and engineered antibodies, and fragments thereof.

Exemplary functional antibody fragments comprising whole or essentially whole variable regions of both light and heavy chains are defined as follows:

(i) Fv, defined as a genetically engineered fragment consisting of the variable region of the light chain and the variable region of the heavy chain expressed as two chains;

(ii) single-chain Fv (“scFv”), a genetically engineered single-chain molecule including the variable region of the light chain and the variable region of the heavy chain, linked by a suitable polypeptide linker.

(iii) Fab, a fragment of an antibody molecule containing a monovalent antigen-binding portion of an antibody molecule, obtained by treating whole antibody with the enzyme papain to yield the intact light chain and the Fd fragment of the heavy chain, which consists of the variable and CHI domains thereof;

(iv) Fab′, a fragment of an antibody molecule containing a monovalent antigen-binding portion of an antibody molecule, obtained by treating whole antibody with the enzyme pepsin, followed by reduction (two Fab′ fragments are obtained per antibody molecule); and

(v) F(ab′)2, a fragment of an antibody molecule containing a monovalent antigen-binding portion of an antibody molecule, obtained by treating whole antibody with the enzyme pepsin (i.e., a dimer of Fab′ fragments held together by two disulfide bonds).

The term “antigen” as used herein is a molecule or a portion of a molecule capable of being bound by an antibody. The antigen is typically capable of inducing an animal to produce antibody capable of binding to an epitope of that antigen. An antigen may have one or more epitopes. The specific reaction referred to above is meant to indicate that the antigen will react, in a highly selective manner, with its corresponding antibody and not with the multitude of other antibodies which may be evoked by other antigens. An “antigenic peptide” is a peptide which is capable of specifically binding an antibody.

In another embodiment, detection of the capacity of an antibody to specifically bind an antigen probe may be performed by quantifying specific antigen-antibody complex formation. The term “specifically bind” as used herein means that the binding of an antibody to an antigen probe is not competitively inhibited by the presence of non-related molecules.

In certain embodiments, the method of the present invention is performed by determining the capacity of an antigen of the invention to specifically bind antibodies of the IgG isotype, and/or, antibodies of the IgM, within a sample obtained from a subject.

Methods for obtaining suitable antibody-containing biological samples from a subject are well within the ability of those of skill in the art. Typically, suitable samples comprise whole blood and products derived therefrom, such as plasma and serum. In other embodiments, other antibody-containing samples may be used, e.g. CSF, urine and saliva samples.

Numerous well known fluid collection methods can be utilized to collect the biological sample from the subject in order to perform the methods of the invention.

In accordance with the present invention, any suitable immunoassay can be used with the subject peptides. Such techniques are well known to the ordinarily skilled artisan and have been described in many standard immunology manuals and texts. In certain preferable embodiments, determining the capacity of the antibodies to specifically bind the antigen probes is performed using an antigen probe array-based method. Preferably, the array is incubated with suitably diluted serum of the subject so as to allow specific binding between antibodies contained in the serum and the immobilized antigen probes, washing out unbound serum from the array, incubating the washed array with a detectable label-conjugated ligand of antibodies of the desired isotype, washing out unbound label from the array, and measuring levels of the label bound to each antigen probe.

According to some aspects the methods of the present invention may be practiced using antigen arrays as disclosed in WO 02/08755 and U.S. 2005/0260770 to some of the inventors of the present invention. WO 02/08755 is directed to a system and an article of manufacture for clustering and thereby identifying predefined antigens reactive with undetermined immunoglobulins of sera derived from patient subjects in need of diagnosis of disease or monitoring of treatment. Further disclosed are diagnostic methods, and systems useful in these methods, employing the step of clustering a subset of antigens of a plurality of antigens, said subset of antigens being reactive with a plurality of antibodies being derived from a plurality of patients, and associating or disassociating the antibodies of a subject with the resulting cluster. U.S. Pat. App. Pub. No. 2005/0260770 to some of the inventors of the present invention discloses an antigen array system and diagnostic uses thereof. The application provides a method of diagnosing an immune disease, particularly diabetes type 1, or a predisposition thereto in a subject, comprising determining a capacity of immunoglobulins of the subject to specifically bind each antigen probe of an antigen probe set. The teachings of said disclosures are incorporated in their entirety as if fully set forth herein.

In other embodiments, various other immunoassays may be used, including, without limitation, enzyme-linked immunosorbent assay (ELISA), flow cytometry with multiplex beads (such as the system made by Luminex), surface plasmon resonance (SPR), elipsometry, and various other immunoassays which employ, for example, laser scanning, light detecting, photon detecting via a photo-multiplier, photographing with a digital camera based system or video system, radiation counting, fluorescence detecting, electronic, magnetic detecting and any other system that allows quantitative measurement of antigen-antibody binding.

Various methods have been developed for preparing arrays suitable for the methods of the present invention. State-of-the-art methods involves using a robotic apparatus to apply or “spot” distinct solutions containing antigen probes to closely spaced specific addressable locations on the surface of a planar support, typically a glass support, such as a microscope slide, which is subsequently processed by suitable thermal and/or chemical treatment to attach antigen probes to the surface of the support. Conveniently, the glass surface is first activated by a chemical treatment that leaves a layer of reactive groups such as epoxy groups on the surface, which bind covalently any molecule containing free amine or thiol groups. Suitable supports may also include silicon, nitrocellulose, paper, cellulosic supports and the like.

Preferably, each antigen probe, or distinct subset of antigen probes of the present invention, which is attached to a specific addressable location of the array is attached independently to at least two, more preferably to at least three separate specific addressable locations of the array in order to enable generation of statistically robust data.

In addition to antigen probes of the invention, the array may advantageously include control antigen probes or other standard chemicals. Such control antigen probes may include normalization control probes. The signals obtained from the normalization control probes provide a control for variations in binding conditions, label intensity, “reading” efficiency and other factors that may cause the signal of a given binding antibody-probe ligand interaction to vary. For example, signals, such as fluorescence intensity, read from all other antigen probes of the antigen probe array are divided by the signal (e.g., fluorescence intensity) from the normalization control probes thereby normalizing the measurements. Normalization control probes can be bound to various addressable locations on the antigen probe array to control for spatial variation in antibody-ligand probe efficiency. Preferably, normalization control probes are located at the corners or edges of the array to control for edge effects, as well as in the middle of the array.

The labeled antibody ligands may be of any of various suitable types of antibody ligand. Preferably, the antibody ligand is an antibody which is capable of specifically binding the Fc portion of the antibodies of the subject used. For example, where the antibodies of the subject are of the IgM isotype, the antibody ligand is preferably an antibody capable of specifically binding to the Fc region of IgM antibodies of the subject.

The ligand of the antibodies of the subject may be conjugated to any of various types of detectable labels. Preferably the label is a fluorophore, most preferably Cy3. Alternately, the fluorophore may be any of various fluorophores, including Cy5, fluorescein isothiocyanate (FITC), phycoerythrin (PE), rhodamine, Texas red, and the like. Suitable fluorophore-conjugated antibodies specific for antibodies of a specific isotype are widely available from commercial suppliers and methods of their production are well established.

Antibodies of the subject may be isolated for analysis of their antigen probe binding capacity in any of various ways, depending on the application and purpose. While the subject's antibodies may be suitably and conveniently in the form of blood serum or plasma or a dilution thereof (e.g. 1:10 dilution), the antibodies may be subjected to any desired degree of purification prior to being tested for their capacity to specifically bind antigen probes. The method of the present invention may be practiced using whole antibodies of the subject, or antibody fragments of the subject which comprises an antibody variable region.

Data Analysis

In some embodiments, the methods of the invention may employ the use of learning and pattern recognition analyzers, clustering algorithms and the like, in order to discriminate between reactivity patterns of subjects having a disorder associated with graft reject (e.g., PGD) to control samples. For example, the methods may include determining the reactivity of antibodies in a test sample to a plurality of antigens, and comparing the resulting pattern to the reactivity patterns of negative and positive control samples using such algorithms and/or analyzers.

Thus, in another embodiment, a significant difference between the reactivity pattern of a test sample compared to a reactivity pattern of a control sample, wherein the difference is computed using a learning and pattern recognition algorithm, indicates that the subject is afflicted with having a disorder associated with graft reject. For example, the algorithm may include, without limitation, supervised or non-supervised classifiers including statistical algorithms including, but not limited to, principal component analysis (PCA), partial least squares (PLS), multiple linear regression (MLR), principal component regression (PCR), discriminant function analysis (DFA) including linear discriminant analysis (LDA), and cluster analysis including nearest neighbor, artificial neural networks, coupled two-way clustering algorithms, multi-layer perceptrons (MLP), generalized regression neural network (GRNN), fuzzy inference systems (FIS), self-organizing map (SOM), genetic algorithms (GAS), neuro-fuzzy systems (NFS) and adaptive resonance theory (ART).

In certain embodiments, one or more algorithms or computer programs may be used for comparing the amount of each antibody quantified in the test sample against a predetermined cutoff (or against a number of predetermined cutoffs). Alternatively, one or more instructions for manually performing the necessary steps by a human can be provided.

Algorithms for determining and comparing pattern analysis include, but are not limited to, principal component analysis, Fischer linear analysis, neural network algorithms, genetic algorithms, fuzzy logic pattern recognition, and the like. After analysis is completed, the resulting information can, for example, be displayed on display, transmitted to a host computer, or stored on a storage device for subsequent retrieval.

Many of the algorithms are neural network based algorithms. A neural network has an input layer, processing layers and an output layer. The information in a neural network is distributed throughout the processing layers. The processing layers are made up of nodes that simulate the neurons by the interconnection to their nodes. Similar to statistical analysis revealing underlying patterns in a collection of data, neural networks locate consistent patterns in a collection of data, based on predetermined criteria.

Suitable pattern recognition algorithms include, but are not limited to, principal component analysis (PCA), Fisher linear discriminant analysis (FLDA), soft independent modeling of class analogy (SIMCA), K-nearest neighbors (KNN), neural networks, genetic algorithms, fuzzy logic, and other pattern recognition algorithms. In some embodiments, the Fisher linear discriminant analysis (FLDA) and canonical discriminant analysis (CDA) as well as combinations thereof are used to compare the output signature and the available data from the database.

In other embodiments, principal component analysis is used. Principal component analysis (PCA) involves a mathematical technique that transforms a number of correlated variables into a smaller number of uncorrelated variables. The smaller number of uncorrelated variables is known as principal components. The first principal component or eigenvector accounts for as much of the variability in the data as possible, and each succeeding component accounts for as much of the remaining variability as possible. The main objective of PCA is to reduce the dimensionality of the data set and to identify new underlying variables.

Principal component analysis compares the structure of two or more covariance matrices in a hierarchical fashion. For instance, one matrix might be identical to another except that each element of the matrix is multiplied by a single constant. The matrices are thus proportional to one another. More particularly, the matrices share identical eigenvectors (or principal components), but their eigenvalues differ by a constant. Another relationship between matrices is that they share principal components in common, but their eigenvalues differ. The mathematical technique used in principal component analysis is called eigenanalysis. The eigenvector associated with the largest eigenvalue has the same direction as the first principal component. The eigenvector associated with the second largest eigenvalue determines the direction of the second principal component. The sum of the eigenvalues equals the trace of the square matrix and the maximum number of eigenvectors equals the number of rows of this matrix.

In another embodiment, the algorithm is a classifier. One type of classifier is created by “training” the algorithm with data from the training set and whose performance is evaluated with the test set data. Examples of classifiers used in conjunction with the invention are discriminant analysis, decision tree analysis, receiver operator curves or split and score analysis.

The term “decision tree” refers to a classifier with a flow-chart-like tree structure employed for classification. Decision trees consist of repeated splits of a data set into subsets. Each split consists of a simple rule applied to one variable, e.g., “if value of “variable 1” larger than “threshold 1”; then go left, else go right”. Accordingly, the given feature space is partitioned into a set of rectangles with each rectangle assigned to one class.

The terms “test set” or “unknown” or “validation set” refer to a subset of the entire available data set consisting of those entries not included in the training set. Test data is applied to evaluate classifier performance.

The terms “training set” or “known set” or “reference set” refer to a subset of the respective entire available data set. This subset is typically randomly selected, and is solely used for the purpose of classifier construction.

Organ Transplant Rejection

Post-transplantation complications may include organ rejection, infection, renal insufficiency and in some cases cancer.

Rejection or dysfunction of solid organs may be hyperacute, accelerated, acute, or chronic (late), depending on the onset of graft destruction. Hyperacute rejection is the term applied to very early graft destruction, usually within the first 48 hours. It is humorally mediated and occurs when preformed antibodies are present in the recipient's serum that are specific for donor antigens expressed on graft vascular endothelial cells. Acute rejection has an onset of two days to three months after transplantation and can have humoral and/or cellular mechanisms. Chronic rejection develops months to years after acute rejection episodes have subsided.

While these categories overlap somewhat in timing, they can be distinguished histopathologically. The symptoms vary by organ and are known for one skilled in the art. In lung transplantation, signs for hyperacute rejection include poor oxygenation, fever and cough; signs for accelerated rejection include decreased FEV₁ (forced expiratory volume in 1 sec); signs for acute rejection include infiltrate (seen on x-ray), interstitial perivascular, infiltrate (detected by transbronchial biopsy) and decreased FEV 1; and signs for chronic rejection include obliterative bronchiolitis, cough and dyspnea.

Hyperacute rejection occurs within 48 h of transplantation and is caused by preexisting complement-fixing antibodies to graft antigens (presensitization). It has become rare (1%) as pretransplantation screening has improved. Hyperacute rejection is characterized by small-vessel thrombosis and graft infarction. No treatment is effective except graft removal.

Accelerated rejection occurs 3 to 5 days after transplantation and is caused by preexisting noncomplement-fixing antibodies to graft antigens. Accelerated rejection is also rare. It is characterized histopathologically by cellular infiltrate with or without vascular changes. Treatment is with high-dose pulse corticosteroids or, if vascular changes occur, antilymphocyte preparations. Plasmapheresis, which may clear circulating antibodies more rapidly, has been used.

Acute rejection is graft destruction after transplantation and is caused by a T cell-mediated delayed hypersensitivity reaction to allograft histocompatibility antigens. It accounts for about half of all rejection episodes that occur within 10 yr. Acute rejection is characterized by mononuclear cellular infiltration, with varying degrees of hemorrhage, edema, and necrosis. Vascular integrity is usually maintained, although vascular endothelium appears to be a primary target. Acute rejection is often reversed by intensifying immunosuppressive therapy (e.g., with pulse corticosteroids, ALG, or both). After rejection reversal, severely damaged parts of the graft heal by fibrosis, the remainder of the graft functions normally, immunosuppressant doses can be reduced to very low levels, and the allograft can survive for long periods.

Chronic rejection is graft dysfunction, often without fever, typically occurring months to years after transplantation but sometimes within weeks. Causes are multiple and include early antibody-mediated rejection, periprocedural ischemia and reperfusion injury, drug toxicity, infection, and vascular factors (e.g., hypertension, hyperlipidemia). Chronic rejection accounts for most of the other half of all rejection episodes. Proliferation of neointima consisting of smooth muscle cells and extracellular matrix (transplantation atherosclerosis) gradually and eventually occludes vessel lumina, resulting in patchy ischemia and fibrosis of the graft. Chronic rejection progresses insidiously despite immunosuppressive therapy.

The following examples are presented in order to more fully illustrate some embodiments of the invention. They should, in no way be construed, however, as limiting the broad scope of the invention.

### EXAMPLES

### Materials and Methods

Autoantibody Profiling Data

Patients attending scheduled visits during a half-year period in the out-patient clinic at the Danish National Lung Transplant Programme, were included in the study. The transplant program has been described in detail previously (Hagedorn et al., 2010; Burton et al., 2005). For 39 patients, PGD could be evaluated retrospectively from chest radiographs and oxygenation data pertaining to the first 72 postoperative hours. Table 2 below presents clinical characteristics for this patient cohort.

An additional 9 patients for which reactivity data was also available, but original chest radiographs had been discarded were set aside for validation. In this validation cohort, the presence or absence of PGD was ascertained from patient journals (which included day-to-day observations from chest radiographs describing the presence or absence of pulmonary edema and/or infiltrates during the first 72 hours as well as documentation for treatment with nasal oxygen when this had been used).

Reactivity data for immunoglobulin G (IgG) and IgM antibody binding in sera from these patients were retrieved from www.nanotech.dtu.dk/Research/Theory/Stochastic/Research/LungTransplant.aspx.

Antigen microarray preparation, incubation of serum and fluorescent anti-IgG and anti-IgM antibodies, laser scanning, and data preprocessing have been described previously (Hagedorn et al., 2010). Briefly, 504 antigens were judged positive for IgG antibody binding (signal-to-noise ratio above 2 in at least 4 patients) and 610 antigens for IgM antibody binding (473 antigens overlapping). These antigens cover 272 recombinant proteins and synthetic peptides from the sequences of key proteins. The log 2-transformed, median centered, measured intensity of an antigen is denoted the reactivity of the antigen.

Transcript Profiling Data

Data from two gene expression studies (GSE8021 and GSE9102) (Ray et al., 2007; Anraku et al., 2008) were retrieved from the Gene Expression Omnibus database (Barrett et al., 2007). Both studies contrasted samples from donor lungs that later developed PGD against donor lungs that did not. For the GSE9102 study, cDNA microarray data as preprocessed by the authors was used, and covered 6727 Ensembl build 55 human genes (jul2009.archive.ensembl.org). When several probes were available for the same gene, the probe displaying the most significant differential expression were selected to represent that gene. For the GSE8021 study, the original raw data was processed as follows. Affymetrix Human Genome U133A 2.0 Array probes were remapped to 11894 different Ensembl build 55 human genes (Dai et al., 2005). Using these redefined probe-sets, probe intensities were summarized and made comparable between arrays by quantile normalization as implemented in the Robust Multi-Array Average expression measure (Irizarry et al., 2003). It was possible to identify corresponding gene expression for 242 of the 272 proteins on the antigen microarray (89%).

Identification of Differentially Reactive Proteins and Differentially Expressed Genes

For each antigen and detection antibody, differential reactivity between patients without PGD (n=19) and patients with PGD (n=20), was evaluated by calculating ratios (fold-changes), t-statistics and P-values. For each gene measured, differential expression between donor lungs developing PGD (16 and 10) and those that did not (34 and 16) were similarly evaluated by ratios, t-statistics and P-values. Multiple testing was controlled using the False Discovery Rate (FDR) (Benjamini and Hochberg, 1995).

Constructing a High-Confidence Network of Human Protein Interactions

A human protein interaction network was created by pooling human interaction data from several of the largest databases (Lage et al. 2007). Coverage was further increased by transferring data from model organisms. A network-wide confidence score for all interactions, based on network topology, experimental type, and interaction reproducibility, was then established. The reliability of this score as a measure of interaction confidence was confirmed by fitting a calibration curve of the score against a high-confidence set of about 35,000 human interactions. As described in Hagedorn et al., 2010, all interactions with a confidence score above 0.154 were included, resulting in a network containing ˜154,000 unique interactions between ˜12,500 human proteins. Out of the 272 proteins on the antigen microarray, 260 (96%) were among these.

Significance and Biological Themes of Networks

The statistical significance of the number of proteins in a network (the size) extracted from a given larger set of proteins, was estimated by randomly selecting sets of proteins of the same size, each time recording the size of the largest network possible to extract (as described in Hagedorn et al., 2010). For 107 such randomizations, the proportion of random sets of proteins for which equally sized or larger networks could be extracted, establishes the P-value of the network extracted from the original protein set. Over-represented biological processes among proteins in networks were identified by hypergeometric testing of gene ontology terms.

### Example 1

### Antibody Reactivities Reflect PGD Grade

Out of the 48 patients for which IgG and IgM reactivity data was available (Hagedorn et al., 2010), 39 patients were graded according to PGD using chest radiographs and oxygenation data. In this cohort, each antigen included was tested for differential reactivity between patients having had PGD (n=20) and patients without PGD (n=19) by t-testing. The baseline clinical characteristics of the two groups were well matched except that there were a higher proportion of female donors in the PGD group than in the group without PGD (see Table 2; Comparison of clinical parameters and PGD grades).

TABLE 2
Clinical characteristics of patients.

| | All | PGD 0 | PGD 1 | |
|---|---|---|---|---|
| | (n = 39) | (n = 19) | (n = 20) | P-value |
| Recipient age (years) | | | | |
| <40 | 5 | 1 | 4 | ns |
| 40-49 | 5 | 2 | 3 | |
| 50-59 | 13 | 9 | 4 | |
| 60-69 | 12 | 6 | 6 | |
| ≧70 | 4 | 1 | 3 | |
| Recipient sex | | | | |
| Male | 20 | 13 | 7 | 0.06 |
| Female | 19 | 6 | 13 | |
| Donor age (years) | | | | |
| <20 | 5 | 3 | 2 | ns |
| 20-29 | 7 | 3 | 4 | |
| 30-39 | 7 | 5 | 2 | |
| 40-49 | 11 | 4 | 7 | |
| ≧50 | 9 | 4 | 5 | |
| Donor sex | | | | |
| Male | 23 | 16 | 7 | 0.003 |
| Female | 16 | 3 | 13 | |
| Primary diagnosis | | | | |
| COPD | 14 | 6 | 8 | ns |
| A1AT | 15 | 10 | 5 | |
| CF | 6 | 2 | 4 | |
| Other | 4 | 1 | 3 | |
| Antihypertensive | | | | |
| treatment | | | | |
| + | 35 | 17 | 18 | ns |
| − | 4 | 2 | 2 | |
| Number of treated | | | | |
| rejections >A1 | | | | |
| 0 | 6 | 1 | 5 | ns |
| 1 | 7 | 4 | 3 | |
| 2 | 9 | 3 | 6 | |
| 3 | 7 | 4 | 3 | |
| ≧4 | 10 | 7 | 3 | |
| BOS grade | | | | |
| 0, 0-p, 1 | 24 | 14 | 10 | ns |
| 2, 3 | 15 | 5 | 10 | |
| Months post Tx | | | | |
| Average | 72 | 79 | 66 | ns |

ns: P ≧ 0.1

For the 473 antigens for which both IgG and IgM reactivity were detected, the increase or decrease in reactivity between patients that developed PGD and patients that did not was compared (IgG change versus IgM change). Those antigens where the IgG and IgM reactivity changed in the same direction were said to display concordant changes. By ordering the 473 antigens based on the significance of the reactivity changes (lowest P-values comes first), and counting the number of concordant antigens in a sliding window of size 114 (25% of all antigens), it was seen that the lower the P-values, the more antigens displayed concordant reactivity changes. There are two P-values for each antigen (one for the IgG reactivity change and one for the IgM reactivity change), and the ordering of all antigens is based on the larger of the two for each antigen, so that the least significant reactivity decides the place in the ordering.

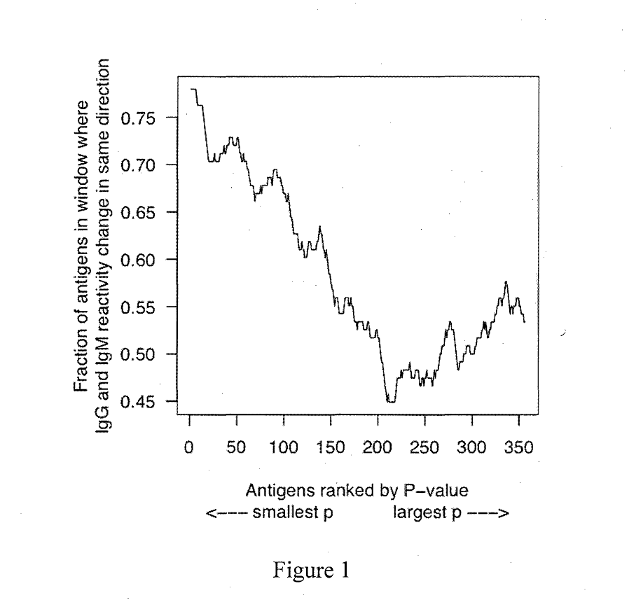

Comparing changes in IgG reactivity with changes in IgM reactivity for each antigen included on the microarray, however, it was observed that the lower the P-values for these changes, the more frequently they changed in the same direction (see FIG. 1).

At a significance threshold of P<0.001 (equal to FDR<0.15), a single antigen, telomerase-associated protein 1 (TEP 1), was identified, displaying four-fold increased reactivity in patients with PGD.

Requiring P<0.05 for the differential reactivity of both IgG and IgM, 16 different proteins (corresponding to 46 different antigens, since several peptides from the same protein were usually detected), were identified.

With these significance thresholds, 17 proteins were identified in all (Table 3). For each protein, the reactivity changes listed are for the most significant antigen identified.

The 17 proteins displaying significant IgG and/or IgM reactivity changes between patients having developed PGD compared to those that did not are listed. For each protein, the log₂ transformed reactivity ratio and P-value (t-test) for the most significant antigen is shown. Gene expression changes between donor lungs developing PGD compared to those that do not, as measured in two independent studies, are also listed (log₂ transformed expression ratio and P-value from t-test).

TABLE 3
Autoreactivity and expression changes for the significant proteins.

| | IgG | | IgM | | mRNA | | mRNA | |
|---|---|---|---|---|---|---|---|---|
| | reactivity | | reactivity | | GSE8021 | | GSE9102 | |
| Gene | log₂ | | log₂ | | log₂ | | log₂ | |
| Symbol | ratio | P | ratio | P | ratio | P | ratio | P |
| EGFR | 1.73 | 0.0029 | 1.30 | 0.011 | −0.04 | 0.55 | −0.33 | 0.083 |
| MBP | 0.93 | 0.0047 | 0.53 | 0.015 | 0.05 | 0.47 | | |
| MLANA | 0.73 | 0.0027 | 0.88 | 0.0070 | 0.03 | 0.42 | −0.15 | 0.31 |
| MUC1 | 4.36 | 0.024 | 2.09 | 0.045 | 0.02 | 0.88 | | |
| MYCL1 | 2.35 | 0.041 | 0.94 | 0.0057 | 0.14 | 0.064 | 0.32 | 0.090 |
| PLCG1 | 2.03 | 0.018 | 0.86 | 0.018 | −0.04 | 0.53 | | |
| PRKCA | 1.63 | 0.021 | 2.40 | 0.028 | 0.12 | 0.067 | 0.24 | 0.021 |
| HSP90AA1 | 0.91 | 0.0015 | −1.14 | 0.0060 | −0.12 | 0.27 | | |
| IGF1R | 2.98 | 0.013 | −0.58 | 0.018 | −0.16 | 0.33 | | |
| RB1 | 0.73 | 0.035 | −0.67 | 0.019 | −0.06 | 0.59 | | |
| CERK | −0.50 | 0.040 | 0.96 | 0.0035 | 0.16 | 0.098 | 0.02 | 0.87 |
| HSPD1 | −0.66 | 0.0043 | 2.49 | 0.0047 | | | | |
| TEP1 | −1.40 | 0.20 | 2.16 | 0.0009 | 0.04 | 0.51 | | |
| CYP3A4 | −1.07 | 0.0084 | −0.52 | 0.026 | 0.03 | 0.59 | | |
| SOCS3 | −0.47 | 0.0065 | −0.83 | 0.023 | −0.27 | 0.17 | −0.56 | 0.050 |
| TARP | −0.37 | 0.0013 | 1.37 | 0.013 | | | | |
| TP53 | −0.56 | 0.028 | −0.60 | 0.049 | −0.01 | 0.97 | | |

Out of the 17 proteins identified in this manner, 6 proteins (HSPD1, HSP90AA1, IGF1R, PRKCA, TARP, and TP53) were previously found to be differentially reactive in connection with BOS (Hagedorn et al., 2010).

Two-factor analysis of variance (ANOVA) for these proteins, with PGD and BOS as the factors, still identified all proteins except TP53 (P=0.11) as displaying significant differences for PGD (P<0.05), see Table 4 listing the resulting P-values for each detection IgG and IgM antibodies for BOS and PGD and FIG. 2 for distributions of reactivities for the 6 antigens.

TABLE 4
Analysis of autoreactivities including both BOS and PGD status.

| | P | P | P | P |
|---|---|---|---|---|
| Gene symbol | (BOS IgG) | (PGD IgG) | (BOS IgM) | (PGD IgM) |
| HSPD1 | 0.031 | 0.016 | 0.064 | 0.0087 |
| HSP90AA1 | 0.10 | 0.0042 | 0.048 | 0.016 |
| IGF1R | 0.18 | 0.020 | 0.050 | 0.050 |
| PRKCA | 0.041 | 0.049 | 0.28 | 0.040 |
| TARP | 0.032 | 0.0052 | 0.0085 | 0.030 |
| TP53 | 0.021 | 0.090 | 0.095 | 0.11 |

### Example 2

### PGD Profile is Organized in a Specific Protein Interaction Network

The known interactions between the 17 proteins that displayed significant differential autoantibody reactivity (Table 3) were analyzed. This allowed the examination of whether the informative antigens formed networks with specific biological functions. Other large-scale data integrative methods have shown that well-defined interaction networks can often be functionally related to pathological processes and complex diseases (Hagedorn et al., 2010; Lage et al., 2007).

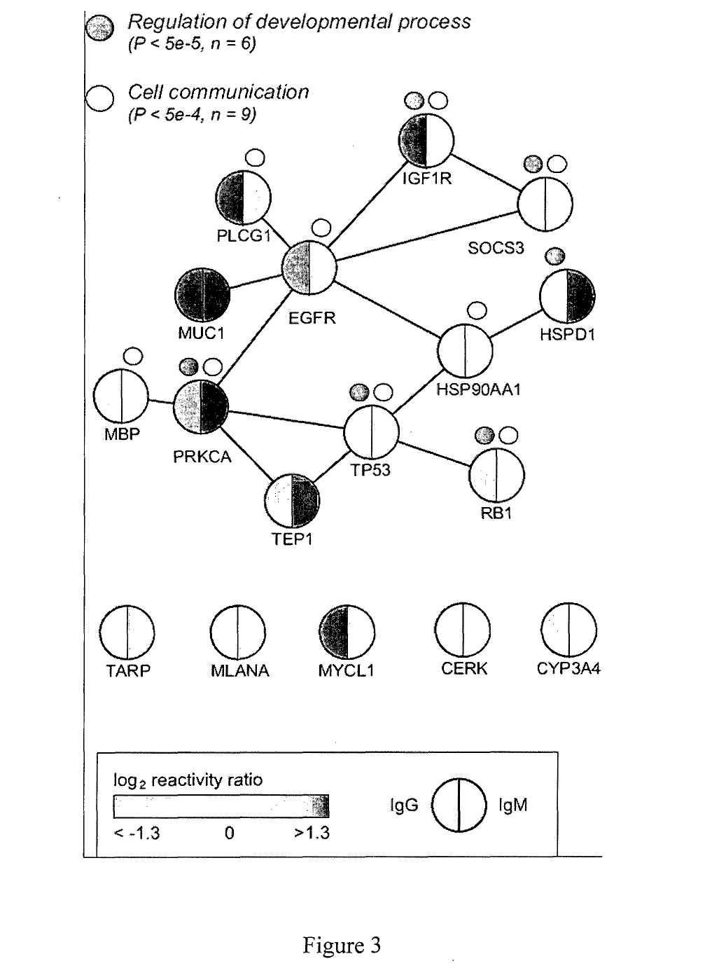

For 15 of the 17 proteins, interaction data was available. An interconnected network consisting of 12 proteins was identified, which is significantly more than would be expected by chance (P=3×10⁻⁶) as determined by randomly selecting 15 proteins out of the 260 proteins on the array where interaction data is available, recording the largest interconnected network possible to construct from these, and repeating this 107 times. Also shown in FIG. 3 are the results of hypergeometric testing on the gene ontology biological process terms assigned to the proteins in the network. Regulation of developmental process (P<5×10⁻⁵) and cell communication (P<5×10⁻⁴) were two of the most significantly enriched terms.

The biological meaning of the profile of autoreactive proteins was extended by integrating information about interactions between the proteins as well as their functional roles. Indeed, out of the 17 proteins identified, 12 proteins could be organized in a network with a distinct biological profile involved in regulation of development and cellular communication (FIG. 3), both of which play a role in coordinating cellular proliferation. Comparing with expression levels in donor lungs as measured in two already published studies (Ray et al., 2007, Anraku et al., 2008) for the genes encoding 15 of the 17 proteins, a significant positive correlation with autoreactivity changes in the recipients was observed. This correlation was observed even though the gene expressions and autoreactivity were measured in different patient cohorts.

The interpretation of these correlated molecular events with respect to PGD is not straightforward. Downstream signaling from both EGFR and IGF1R, which are central components in the protein network in FIG. 3, typically includes activation of the mitogen-activated protein kinase cascade and subsequent transcriptional activation of immediate-early genes such as the activating protein 1 (AP-1) transcription factor subunits FOS and JUN (Hess et al. 2004). Indeed, AP-1 is known to regulate processes such as proliferation and transformation, which meshes well with the biological profile of the identified proteins (FIG. 3). Interrogation of FOS and JUN gene expression in the GSE8021 study showed that FOS display almost two-fold lower expression and JUN 1.2-fold lower expression in donor lungs that later developed PGD compared to those that did not (both with P<0.05).

In clinical studies with lung biopsies, PGD has been associated with acute alveolar damage early and fibrosis later, leading to reduced lung volumes (Burton et al., 2007). The fibrotic response in inflamed airways most probably manifests itself in part by increased airway epithelial cell proliferation rates (Leigh et al., 1995). It is hypothesize that such aberrant proliferation may in part be caused by growth factor mediated, proliferative, signaling in the donor lung not in balance with the surrounding tissues and organs in the recipient, inferred by the differences in gene expression that correlates with altered autoreactivity against the encoded proteins.

### Example 3

### PGD Profile can be Used to Predict PGD Status in an Independent Patient Cohort

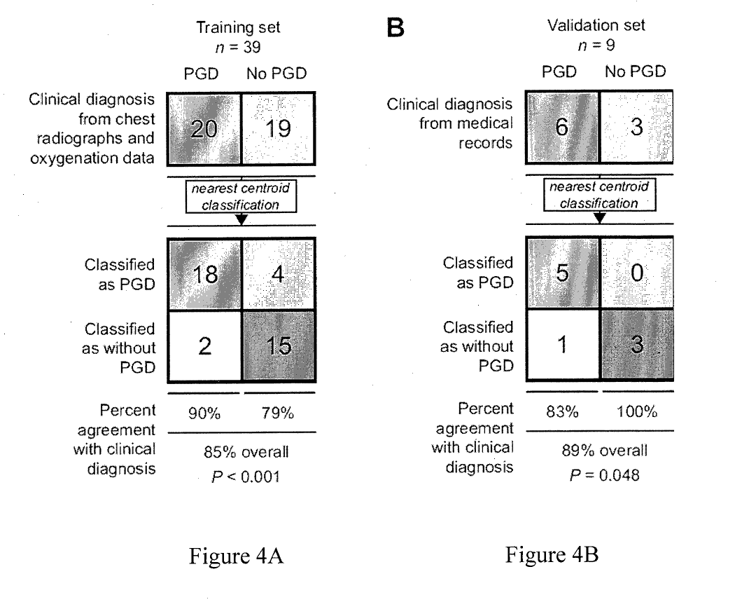

In the validation cohort of 9 patients, 6 had PGD grade 1, and for the remaining 3 there is no evidence to suggest PGD. All patients were extubated in the first 24 hours and none qualified for a PGD grade 2 or higher. A nearest centroid classifier (Hastie et al., Springer Verlag, New York; 2001) was constructed from the 17 differentially reactive proteins identified (FIG. 4A), and used to predict the PGD grades of the 9 patients in this validation cohort (FIG. 4B). Here, 5 out of 6 patients having had PGD were correctly identified (83% sensitivity), and all 3 patients without PGD were classified as such (100% specificity), giving an overall classification accuracy of 89% (P=0.048 by Fisher's exact test). This is comparable to the classification accuracy in the test set (85%).

### Example 4

### Identifying Transcript Differences for the Proteins with Altered Reactivity

Two recent studies have investigated gene expression differences in donor lungs developing PGD (Ray et al., 2007; Anraku et al., 2008). Differential gene expression in each study was evaluated by t-testing. Out of the 17 differentially reactive proteins identified, 15 proteins could be paired with gene expression in the Ray et al., study, and 6 with expressions from the Anraku study (Table 3).

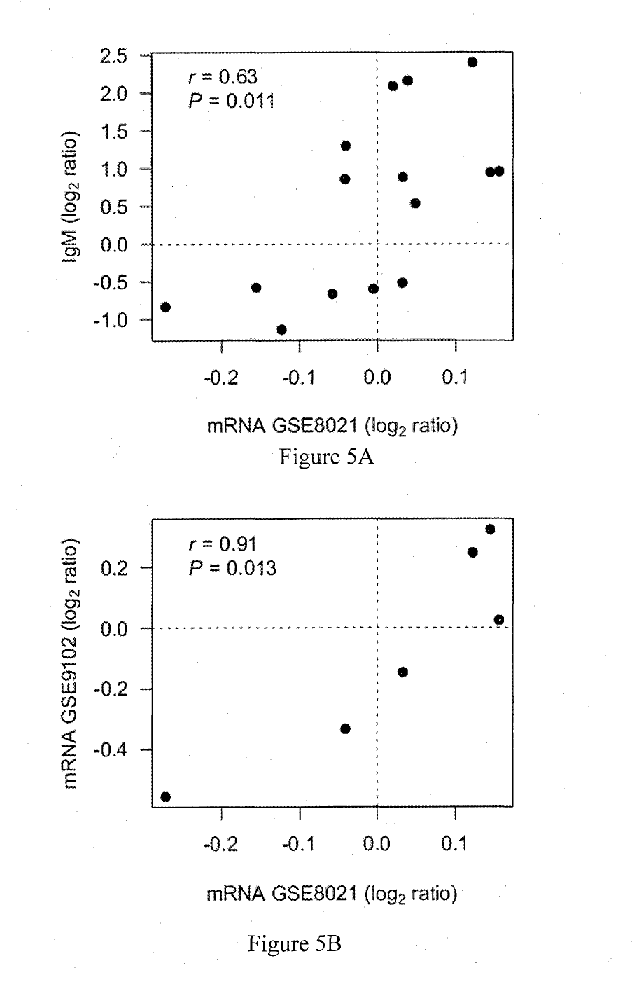

Comparing differences in IgM reactivity with differences in gene expression levels in the first study (study GSE8021 in Table 3), 12 out of 15 change in the same direction (80% concordance, P=0.04 by Fisher's Exact Test), i.e. increased expression is significantly associated with increased reactivity and vice versa. The same conclusion is reached when calculating Pearson's product-moment correlation (r=0.63, P=0.011), see FIG. 5A. For IgG reactivity, no significant correlation with gene expression changes was observed (r=−0.01, P=0.98).

Inspection of the P-values for the differential expressions (study GSE8021 in Table 3) show that none of them have P<0.05, which is usually a standard threshold of significance. Still, 5 out of 6 genes display the same direction as well as magnitude of change when comparing with the second gene expression study (GSE9102 in Table 3) which is a significant correlation (r=0.91, P=0.013), see FIG. 5B.

The link between donor transcript levels and recipient autoantibody repertoires reported here is supported by significant statistical results on four biological levels: at the level of autoreactive protein selection, at the level of network size and biological process overrepresentation, at the level of classification accuracy in an independent validation cohort of 9 patients, and at the level of correlation with gene expression changes in two other independent patient cohorts of 50 and 26 patients respectively (Ray et al., 2007, Anraku et al., 2008). Even random selections of 17 proteins out of the 273 present on the antigen microarray, not requiring significant differential reactivity, network size, or discriminatory power, only achieves equal or higher correlation with gene expression changes compared to that achieved by the 17 proteins reported in this study (r≧0.63) in 16 out of 1000 attempts (P=0.016), confirming its significance.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

### REFERENCES

Christie et al. J Heart Lung Transplant 2005; 24:1454-1459.
Arcasoy et al., J Heart Lung Transplant 2005; 24: 1483-1488.
Oto et al., Am J Transpl 2008; 8: 2132-9
Lee and Christie. Proc Am Thorac Soc 2009; 6: 39-46.
Pelaez et al., Am J Transpl 2010; 10: 900-907.
Salama et al., Am J Transplant 2010; 10: 628-636.
Bobadilla et al., Am J Respir Crit. Care Med 2008 177:660-8.
Sumpter and Wilkes, Am J Physiol Lung Cell Mol Physiol 2004; 286: L1129-L1139.
Hagedorn et al., Immunology 2010; 130(3):427-435.
Ray et al., Am J Transplant 2007; 7: 2396-2405.
Anraku et al., Am J Transplant 2008; 8:2140-2148.
Cohen I R. Immunol Today 1992; 13: 490-494.
Burton et al., J Heart Lung Transplant 2005; 24: 1834-1843.
Barrett et al. Nucleic Acids Res 2007; 35:D760-D765.
Dai et al., Nucleic Acids Res 2005 33:e175.
Irizarry et al., Nucleic Acids Res 2003 31:e15.
Benjamini and Hochberg, J R Stat Soc 1995 57: 289-300.
Lage et al. Nat Biotechnol 2007; 25: 309-316.
Hastie et al., Springer Verlag, New York; 2001
Bharat et al., Ann Thorac Surg 2008; 86: 189-195
Hess et al. J Cell Sci 2004; 117: 5965-5973.
Burton et al., Eur J Cardiothorac Surg 2007; 31: 75-82.
Leigh et al., Am J Respir Cell Mol Biol 1995; 12: 605-612.
Quintana et al. Lupus, 2006; 15: 428-430.
Merbl, et al. J Clin Invest., 2007 March; 117(3):712-8.
Cohen I R. J. Autoimmun. 2007 December; 29(4):246-9
Li Z G, et al. Lupus. 2005; 14(11):884-9.
Hueber et al. Arthritis Rheum. 2005 September; 52(9):2645-55.
Lalive et al. Neurology. 2006 Jul. 11; 67(1):176-7.

1. A method of diagnosing primary graft dysfunction in a subject in need thereof, the method comprising determining the reactivity of antibodies in a sample obtained from the subject to a plurality of antigens selected from the group consisting of: TEP1, EGFR, MBP, MLANA, MUC1, MYCL1, PLCG1, RB1, CERK, CYP3A4, SOC3, PRKCA, HSP90AA1, IGF1R, HSPD1, TARP and TP53, thereby determining the reactivity pattern of the sample to the plurality of antigens, and comparing said reactivity pattern of the sample to a control reactivity pattern, wherein a significant difference between said reactivity pattern of the sample compared to the control reactivity pattern is an indication that the subject is afflicted with primary graft dysfunction.

2. The method of claim 1, wherein the graft is selected from the group consisting of: lung, heart, kidney and liver.

3. The method of claim 1, wherein the graft is a lung.

4. The method of claim 1, wherein the antibodies are selected from immunoglobulin G (IgG) and IgM antibodies.

5. The method of claim 1, wherein the plurality of antigens comprises at least three different antigens, at least four different antigens, at least five different antigens, at least ten different antigens or at least fifteen different antigens.

6-9. (canceled)

10. The method of claim 1, wherein the plurality of antigens comprises TEP1, EGFR, MBP, MLANA, MUC1, MYCL1, PLCG1, RB1, CERK, CYP3A4, SOC3, PRKCA, HSP90AA1, IGF1R, HSPD1, TARP and TP53.

11. The method of claim 1, wherein the plurality of antigens comprises no more than about 30 antigens.

12. A method for diagnosing a condition associated with organ transplantation rejection in a subject in need thereof, the method comprising determining the reactivity of antibodies in a sample obtained from the subject to a plurality of antigens selected from the group consisting of: TEP1, EGFR, MBP, MLANA, MUC1, MYCL1, PLCG1, RB1, CERK, CYP3A4 and SOC3, thereby determining the reactivity pattern of the sample to the plurality of antigens, and comparing said reactivity pattern of the sample to a control reactivity pattern, wherein a significant difference between said reactivity pattern of the sample compared to the control reactivity pattern is an indication that the subject is afflicted with a disorder or condition associated with transplantation rejection.

13. The method of claim 12, wherein said organ is selected from the group consisting of: lung, heart, kidney and liver.

14. The method of claim 12, wherein said organ is lung.

15. The method of claim 12, wherein the condition associated with organ transplantation rejection is primary graft dysfunction.

16. The method of claim 12, wherein the antibodies are selected from IgG and IgM antibodies.

17. The method of claim 12, wherein the plurality of antigens comprises at least 3 different antigens, at least 4 different antigen or at least 5 different antigens.

18-19. (canceled)

20. The method of claim 12, wherein the plurality of antigens comprises TEP1, EGFR, MBP, MLANA, MUC1, MYCL1, PLCG1, RB1, CERK, CYP3A4 and SOC3.

21. The method of claim 12, wherein the plurality of antigens further comprises PRKCA, HSP90AA1, IGF1R, HSPD1, TARP and TP53.

22. The method of claim 1, wherein the control is selected from the group consisting of a sample from at least one individual, a panel of control samples from a set of individuals, and a stored set of data from control individuals.

23. The method of claim 1, wherein the control reactivity pattern is selected from the group consisting of: a control reactivity pattern obtained from said subject before undergoing organ transplantation, a control reactivity pattern obtained from healthy subjects, a control reactivity pattern obtained from transplant recipients who did not develop a disorder or condition associated with transplantation rejection or a control reactivity pattern obtained from transplant recipients who did not develop PGD.

24-26. (canceled)

27. The method of claim 1, wherein the sample is a serum sample.

28. The method of claim 1, wherein said plurality of antigens is used in the form of an antigen array.

29. A kit for the diagnosis primary graft dysfunction comprising a plurality of antigens selected from the group consisting of TEP1, EGFR, MBP, MLANA, MUC1, MYCL1, PLCG1, RB1, CERK, CYP3A4, SOC3, PRKCA, HSP90AA1, IGF1R, HSPD1, TARP and TP53.

30. A kit for the diagnosis of a condition associated with organ transplantation rejection comprising a plurality of antigens selected from the group consisting of TEP1, EGFR, MBP, MLANA, MUC1, MYCL1, PLCG1, RB1, CERK, CYP3A4 and SOC3.

31. The kit of claim 29, wherein said kit is in the form of an antigen array or wherein the kit further comprises means selected from means for determining the reactivity of antibodies in a sample to the plurality of antigens or means for comparing reactivity patterns of antibodies in different samples to the plurality of antigens.

32. The kit of claim 30, wherein said kit is in the form of an antigen array or wherein the kit further comprises means selected from means for determining the reactivity of antibodies in a sample to the plurality of antigens or means for comparing reactivity patterns of antibodies in different samples to the plurality of antigens.

33. (canceled)

34. An antigen probe set comprising a plurality of antigen probes selected from the group consisting of TEP1, EGFR, MBP, MLANA, MUC1, MYCL1, PLCG1, RB1, CERK, CYP3A4, SOC3, PRKCA, HSP90AA1, IGF1R, HSPD1, TARP and TP53.

35. The antigen probe set of claim 34 for use in diagnosing primary graft dysfunction in a subject in need thereof.


SEQUENCE LISTING (17 sequences)

SEQ ID NO: 1 - protein, 2627 aa, Homo sapiens
MEKLHGHVSAHPDILSLENRCLAMLPDLQPLEKLHQHVSTHSDILSLKNQCLATLPDLKTMEKPHGYVSAHPDILSLENQCLATLSDLKTMEKPHGHVSAHPDILSLENRCLATLSSLKSTVSASPLFQSLQISHMTQADLYRVNNSNCLLSEPPSWRAQHFSKGLDLSTCPIALKSISATETAQEATLGRWFDSEEKKGAETQMPSYSLSLGEEEEVEDLAVKLTSGDSESHPEPTDHVLQEKKMALLSLLCSTLVSEVNMNNTSDPTLAAIFEICRELALLEPEFILKASLYARQQLNVRNVANNILAIAAFLPACRPHLRRYFCAIVQLPSDWIQVAELYQSLAEGDKNKLVPLPACLRTAMTDKFAQFDEYQLAKYNPRKHRAKRHPRRPPRSPGMEPPFSHRCFPRYIGFLREEQRKFEKAGDTVSEKKNPPRFTLKKLVQRLHIHKPAQHVQALLGYRYPSNLQLFSRSRLPGPWDSSRAGKRMKLSRPETWERELSLRGNKASVWEELIENGKLPFMAMLRNLCNLLRVGISSRHHELILQRLQHAKSVIHSRQFPFRFLNAHDAIDALEAQLRNQALPFPSNITLMRRILTRNEKNRPRRRFLCHLSRQQLRMAMRIPVLYEQLKREKLRVHKARQWKYDGEMLNRYRQALETAVNLSVKHSLPLLPGRTVLVYLTDANADRLCPKSNPQGPPLNYALLLIGMMITRAEQVDVVLCGGDTLKTAVLKAEEGILKTAIKLQAQVQEFDENDGWSLNTFGKYLLSLAGQRVPVDRVILLGQSMDDGMINVAKQLYWQRVNSKCLFVGILLRRVQYLSTDLNPNDVTLSGCTDAILKFIAEHGASHLLEHVGQMDKIFKIPPPPGKTGVQSLRPLEEDTPSPLAPVSQQGWRSIRLFISSTFRDMHGERDLLLRSVLPALQARAAPHRISLHGIDLRWGVTEEETRRNRQLEVCLGEVENAQLFVGILGSRYGYIPPSYNLPDHPHFHWAQQYPSGRSVTEMEVMQFLNRNQRLQPSAQALIYFRDSSFLSSVPDAWKSDFVSESEEAARRISELKSYLSRQKGITCRRYPCEWGGVAAGRPYVGGLEEFGQLVLQDVWNMIQKLYLQPGALLEQPVSIPDDDLVQATFQQLQKPPSPARPRLLQDTVQRLMLPHGRLSLVTGQSGQGKTAFLASLVSALQAPDGAKVASLVFFHFSGARPDQGLALTLLRRLCTYLRGQLKEPGALPSTYRSLVWELQQRLLPKSAESLHPGQTQVLIIDGADRLVDQNGQLISDWIPKKLPRCVHLVLSVSSDAGLGETLEQSQGAHVLALGPLEASARARLVREELALYGKRLEESPFNNQMRLLLVKRESGRPLYLRLVTDHLRLFTLYEQVSERLRTLPATVPLLLQHILSTLEKEHGPDVLPQALTALEVTRSGLTVDQLHGVLSVWRTLPKGTKSWEEAVAAGNSGDPYPMGPFACLVQSLRSLLGEGPLERPGARLCLPDGPLRTAAKRCYGKRPGLEDTAHILIAAQLWKTCDADASGTFRSCPPEALGDLPYHLLQSGNRGLLSKFLTNLHVVAAHLELGLVSRLLEAHALYASSVPKEEQKLPEADVAVFRTFLRQQASILSQYPRLLPQQAANQPLDSPLCHQASLLSRRWHLQHTLRWLNKPRTMKNQQSSSLSLAVSSSPTAVAFSTNGQRAAVGTANGTVYLLDLRTWQEEKSVVSGCDGISACLFLSDDTLFLTAFDGLLELWDLQHGCRVLQTKAHQYQITGCCLSPDCRLLATVCLGGCLKLWDTVRGQLAFQHTYPKSLNCVAFHPEGQVIATGSWAGSISFFQVDGLKVTKDLGAPGASIRTLAFNVPGGVVAVGRLDSMVELWAWREGARLAAFPAHHGFVAAALFLHAGCQLLTAGEDGKVQVWSGSLGRPRGHLGSLSLSPALSVALSPDGDRVAVGYRADGIRIYKISSGSQGAQGQALDVAVSALAWLSPKVLVSGAEDGSLQGWALKECSLQSLWLLSRFQKPVLGLATSQELLASASEDFTVQLWPRQLLTRPHKAEDFPCGTELRGHEGPVSCCSFSTDGGSLATGGRDRSLLCWDVRTPKTPVLIHSFPACHRDWVTGCAWTKDNLLISCSSDGSVGLWDPESGQRLGQFLGHQSAVSAVAAVEEHVVSVSRDGTLKVWDHQGVELTSIPAHSGPISHCAAAMEPRAAGQPGSELLVVTVGLDGATRLWHPLLVCQTHTLLGHSGPVRAAAVSETSGLMLTASEDGSVRLWQVPKEADDTCIPRSSAAVTAVAWAPDGSMAVSGNQAGELILWQEAKAVATAQAPGHIGALIWSSAHTFFVLSADEKISEWQVKLRKGSAPGNLSLHLNRILQEDLGVLTSLDWAPDGHFLILAKADLKLLCMKPGDAPSEIWSSYTENPMILSTHKEYGIFVLQPKDPGVLSFLRQKESGEFEERLNFDINLENPSRTLISITQAKPESESSFLCASSDGILWNLAKCSPEGEWTTGNMWQKKANTPETQTPGTDPSTCRESDASMDSDASMDSEPTPHLKTRQRRKIHSGSVTALHVLPELLVTASKDRDVKLWERPSMQLLGLFRCEGSVSCLEPWLGANSTLQLAVGDVQGNVYFLNWE

SEQ ID NO: 2 - protein, 1210 aa, Homo sapiens [sequence omitted]

SEQ ID NO: 3 - protein, 197 aa, Homo sapiens
MASQKRPSQRHGSKYLATASTMDHARHGFLPRHRDTGILDSIGRFFGGDRGAPKRGSGKVPWLKPGRSPLPSHARSQPGLCNMYKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 4 - protein, 118 aa, Homo sapiens
MPREDAHFIYGYPKKGHGHSYTTAEEAAGIGILTVILGVLLLIGCWYCRRRNGYRALMDKSLHVGTQCALTRRCPQEGFDHRDSKVSLQEKNCEPVVPNAPPAYEKLSAEQSPPPYSP

SEQ ID NO: 5 - protein, 273 aa, Homo sapiens
MTPGTQSPFFLLLLLTVLTVVTGSGHASSTPGGEKETSATQRSSVPSSTEKNALSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 6 - protein, 364 aa, Homo sapiens
MDYDSYQHYFYDYDCGEDFYRSTAPSEDIWKKFELVPSPPTSPPWGLGPGAGDPAPGIGPPEPWPGGCTGDEAESRGHSKGWGRNYASIIRRDCMWSGFSARERLERAVSDRLAPGAPRGNPPKASAAPDCTPSLEAGNPAPAAPCPLGEPKTQACSGSESPSDSENEEIDVVTVEKRQSLGIRKPVTITVRADPLDPCMKHFHISIHQQQHNYAARFPPESCSQEEASERGPQEEVLERDAAGEKEDEEDEEIVSPPPVESEAAQSCHPKPVSSDTEDVTKRKNHNFLERKRRNDLRSRFLALRDQVPTLASCSKAPKVVILSKALEYLQALVGAEKRMATEKRQLRCRQQQLQKRIAYLTGY

SEQ ID NO: 7 - protein, 1291 aa, Homo sapiens
MAGAASPCANGCGPGAPSDAEVLHLCRSLEVGTVMTLFYSKKSQRPERKTFQVKLETRQITWSRGADKIEGAIDIREIKEIRPGKTSRDFDRYQEDPAFRPDQSHCFVILYGMEFRLKTLSLQATSEDEVNMWIKGLTWLMEDTLQAPTPLQIERWLRKQFYSVDRNREDRISAKDLKNMLSQVNYRVPNMRFLRERLTDLEQRSGDITYGQFAQLYRSLMYSAQKTMDLPFLEASTLRAGERPELCRVSLPEFQQFLLDYQGELWAVDRLQVQEFMLSFLRDPLREIEEPYFFLDEFVTFLFSKENSVWNSQLDAVCPDTMNNPLSHYWISSSHNTYLTGDQFSSESSLEAYARCLRMGCRCIELDCWDGPDGMPVIYHGHTLTTKIKFSDVLHTIKEHAFVASEYPVILSIEDHCSIAQQRNMAQYFKKVLGDTLLTKPVEISADGLPSPNQLKRKILIKHKKLAEGSAYEEVPTSMMYSENDISNSIKNGILYLEDPVNHEWYPHYFVLTSSKIYYSEETSSDQGNEDEEEPKEVSSSTELHSNEKWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPNSYAISFRAEGKIKHCRVQQEGQTVMLGNSEFDSLVDLISYYEKHPLYRKMKLRYPINEEALEKIGTAEPDYGALYEGRNPGFYVEANPMPTFKCAVKALFDYKAQREDELTFIKSAIIQNVEKQEGGWWRGDYGGKKQLWFPSNYVEEMVNPVALEPEREHLDENSPLGDLLRGVLDVPACQIAIRPEGKNNRLFVFSISMASVAHWSLDVAADSQEELQDWVKKIREVAQTADARLTEGKIMERRKKIALELSELVVYCRPVPFDEEKIGTERACYRDMSSFPETKAEKYVNKAKGKKFLQYNRLQLSRIYPKGQRLDSSNYDPLPMWICGSQLVALNFQTPDKPMQMNQALFMTGRHCGYVLQPSTMRDEAFDPFDKSSLRGLEPCAISIEVLGARHLPKNGRGIVCPFVEIEVAGAEYDSTKQKTEFVVDNGLNPVWPAKPFHFQISNPEFAFLRFVVYEEDMFSDQNFLAQATFPVKGLKTGYRAVPLKNNYSEDLELASLLIKIDIFPAKQENGDLSPFSGTSLRERGSDASGQLFHGRAREGSFESRYQQPFEDFRISQEHLADHFDSRERRAPRRTRVNGDNRL

SEQ ID NO: 8 - protein, 928 aa, Homo sapiens
MPPKTPRKTAATAAAAAAEPPAPPPPPPPEEDPEQDSGPEDLPLVRLEFEETEEPDFTALCQKLKIPDHVRERAWLTWEKVSSVDGVLGGYIQKKKELWGICIFIAAVDLDEMSFTFTELQKNIEISVHKFFNLLKEIDTSTKVDNAMSRLLKKYDVLFALFSKLERTCELIYLTQPSSSISTEINSALVLKVSWITFLLAKGEVLQMEDDLVISFQLMLCVLDYFIKLSPPMLLKEPYKTAVIPINGSPRTPRRGQNRSARIAKQLENDTRIIEVLCKEHECNIDEVKNVYFKNFIPFMNSLGLVTSNGLPEVENLSKRYEEIYLKNKDLDARLFLDHDKTLQTDSIDSFETQRTPRKSNLDEEVNVIPPHTPVRTVMNTIQQLMMILNSASDQPSENLISYFNNCTVNPKESILKRVKDIGYIFKEKFAKAVGQGCVEIGSQRYKLGVRLYYRVMESMLKSEEERLSIQNFSKLLNDNIFHMSLLACALEVVMATYSRSTSQNLDSGTDLSFPWILNVLNLKAFDFYKVIESFIKAEGNLTREMIKHLERCEHRIMESLAWLSDSPLFDLIKQSKDREGPTDHLESACPLNLPLQNNHTAADMYLSPVRSPKKKGSTTRVNSTANAETQATSAFQTQKPLKSTSLSLFYKKVYRLAYLRLNTLCERLLSEHPELEHIIWTLFQHTLQNEYELMRDRHLDQIMMCSMYGICKVKNIDLKFKIIVTAYKDLPHAVQETFKRVLIKEEEYDSIIVFYNSVFMQRLKTNILQYASTRPPTLSPIPHIPRSPYKFPSSPLRIPGGNIYISPLKSPYKISEGLPTPTKMTPRSRILVSIGESFGTSEKFQKINQMVCNSDRVLKRSAEGSNPPKPLKKLRFDIEGSDEADGSKHLPGESKFQQKLAEMTSTRTRMQKQKMNDSMDTSNKEEK

SEQ ID NO: 9 - protein, 537 aa, Homo sapiens
MGATGAAEPLQSVLWVKQQRCAVSLEPARALLRWWRSPGPGAGAPGADACSVPVSEIIAVEETDVHGKHQGSGKWQKMEKPYAFTVHCVKRARRHRWKWAQVTFWCPEEQLCHLWLQTLREMLEKLTSRPKHLLVFINPFGGKGQGKRIYERKVAPLFTLASITTDIIVTEHANQAKETLYEINIDKYDGIVCVGGDGMFSEVLHGLIGRTQRSAGVDQNHPRAVLVPSSLRIGIIPAGSTDCVCYSTVGTSDAETSALHIVVGDSLAMDVSSVHHNSTLLRYSVSLLGYGFYGDIIKDSEKKRWLGLARYDFSGLKTFLSHHCYEGTVSFLPAQHTVGSPRDRKPCRAGCFVCRQSKQQLEEEQKKALYGLEAAEDVEEWQVVCGKFLAINATNMSCACRRSPRGLSPAAHLGDGSSDLILIRKCSRFNFLRFLIRHTNQQDQFDFTFVEVYRVKKFQFTSKHMEDEDSDLKEGGKKRFGHICSSHPSCCCTVSNSSWNCDGEVLHSPAIEVRVHCQLVRLFARGIEENPKPDSHS

SEQ ID NO: 10 - protein, 503 aa, Homo sapiens
MALIPDLAMETWLLLAVSLVLLYLYGTHSHGLFKKLGIPGPTPLPFLGNILSYHKGFCMFDMECHKKYGKVWGFYDGQQPVLAITDPDMIKTVLVKECYSVFTNRRPFGPVGFMKSAISIAEDEEWKRLRSLLSPTFTSGKLKEMVPIIAQYGDVLVRNLRREAETGKPVTLKDVFGAYSMDVITSTSFGVNIDSLNNPQDPFVENTKKLLRFDFLDPFFLSITVFPFLIPILEVLNICVFPREVTNFLRKSVKRMKESRLEDTQKHRVDFLQLMIDSQNSKETESHKALSDLELVAQSIIFIFAGYETTSSVLSFIMYELATHPDVQQKLQEEIDAVLPNKAPPTYDTVLQMEYLDMVVNETLRLFPIAMRLERVCKKDVEINGMFIPKGVVVMIPSYALHRDPKYWTEPEKFLPERFSKKNKDNIDPYIYTPFGSGPRNCIGMRFALMNMKLALIRVLQNFSFKPCKETQIPLKLSLGGLLQPEKPVVLKVESRDGTVSGA

SEQ ID NO: 11 - protein, 225 aa, Homo sapiens
MVTHSKFPAAGMSRPLDTSLRLKTFSSKSEYQLVVNAVRKLQESGFYWSAVTGGEANLLLSAEPAGTFLIRDSSDQRHFFTLSVKTQSGTKNLRIQCEGGSFSLQSDPRSTQPVPRFDCVLKLVHHYMPPPGAPSFPSPPTEPSSEVPEQPSAQPLPGSPPRRAYYIYSGGEKIPLVLSRPLSSNVATLQHLCRKTVNGHLDSYEKVTQLPGPIREFLDQYDAPL

SEQ ID NO: 12 - protein, 672 aa, Homo sapiens
MADVFPGNDSTASQDVANRFARKGALRQKNVHEVKDHKFIARFFKQPTFCSHCTDFIWGFGKQGFQCQVCCFVVHKRCHEFVTFSCPGADKGPDTDDPRSKHKFKIHTYGSPTFCDHCGSLLYGLIHQGMKCDTCDMNVHKQCVINVPSLCGMDHTEKRGRIYLKAEVADEKLHVTVRDAKNLIPMDPNGLSDPYVKLKLIPDPKNESKQKTKTIRSTLNPQWNESFTFKLKPSDKDRRLSVEIWDWDRTTRNDFMGSLSFGVSELMKMPASGWYKLLNQEEGEYYNVPIPEGDEEGNMELRQKFEKAKLGPAGNKVISPSEDRKQPSNNLDRVKLTDFNFLMVLGKGSFGKVMLADRKGTEELYAIKILKKDVVIQDDDVECTMVEKRVLALLDKPPFLTQLHSCFQTVDRLYFVMEYVNGGDLMYHIQQVGKFKEPQAVFYAAEISIGLFFLHKRGIIYRDLKLDNVMLDSEGHIKIADFGMCKEHMMDGVTTRTFCGTPDYIAPEIIAYQPYGKSVDWWAYGVLLYEMLAGQPPFDGEDEDELFQSIMEHNVSYPKSLSKEAVSICKGLMTKHPAKRLGCGPEGERDVREHAFFRRIDWEKLENREIQPPFKPKVCGKGAENFDKFFTRGQPVLTPPDQLVIANIDQSDFEGFSYVNPQFVHPILQSAV

SEQ ID NO: 13 - protein, 854 aa, Homo sapiens
MPPCSGGDGSTPPGPSLRDRDCPAQSAEYPRDRLDPRPGSPSEASSPPFLRSRAPVNWYQEKAQVFLWHLMVSGSTTLLCLWKQPFHVSAFPVTASLAFRQSQGAGQHLYKDLQPFILLRLLMPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

SEQ ID NO: 14 - protein, 1367 aa, Homo sapiens
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 15 - protein, 573 aa, Homo sapiens
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 16 - protein, 58 aa, Homo sapiens
MQMFPPSPLFFFLQLLKQSSRRLEHTFVFLRNFSLMLLRYIGKKRRATRFWDPRRGTP

SEQ ID NO: 17 - protein, 393 aa, Homo sapiens [sequence omitted]